(19) United States
(12) Patent Application Publication
(10) Pub. No.: US 2026/0248988 A1
(43) Pub. Date: Aug. 27, 2026

(54) A BREAST SHIELD FOR A BREAST PUMP

(71) Applicant: Willow Blossom Holdco Limited, London (GB)

(72) Inventors: Thomas Michael MALLOY, Oxford (GB); Jeremy William CROUCH, Malmesbury, Wiltshire (GB)

(21) Appl. No.: 19/107,485

(22) PCT Filed: Aug. 31, 2023
(86) PCT No.: PCT/EP2023/073972

§ 371 Date: Feb. 27, 2025

(30) Foreign Application Priority Data
Aug. 31, 2022 (GB) 2212672.6

(51) Int. Cl. A61M 1/06

(52) U.S. Cl. CPC A61M 1/066; A61M 1/067; A61M 1/06935

(57) ABSTRACT

There is provided a breast shield for a breast pump, comprising a flexible diaphragm comprising a nipple tunnel for 2024/047203 receiving a user's nipple. The breast shield is configured to form a seal with a user's breast and is configured such that when an internal negative pressure is applied to internal walls of the nipple tunnel, the nipple tunnel contracts to a minimum aperture which conforms to the shape of the user's nipple. The nipple tunnel comprises a plurality of expandable lobes wherein each expandable lobe extends radially from a respective offset point from a central longitudinal axis of the breast shield.

WO

### FIELD OF THE INVENTION

The present disclosure relates to a breast pump and, in particular, to a breast shield for a breast pump for expressing human milk.

### BACKGROUND

A breast pump system is a mechanical or electro-mechanical device that extracts milk from the breasts of a lactating person.

Breast pumps for expressing human breast milk are known. A vacuum is used to simulate suction generated by a feeding child. Breast pumps typically include a breast shield to fit over a breast.

Fully integrated wearable breast pump systems are known in the art. In such pump systems, the suction source, power supply and milk container are contained in a single, wearable device and without the need for external components or connections. Such devices can be provided with a substantially breast shaped convex profile so as to fit within a user's bra for discreet pumping, as well as pumping on-the-go without any tethers to electrical sockets or collection stations. The internal breast shield is convex to fit over a breast.

Known breast pump systems may be provided with multiple breast shields of different sizes, to cater for the different physical requirements of different users, in particular different nipple sizes. The user selects the breast shield which provides the best personal fit, whilst the other breast shields are not used. Further, even the breast shield which provides the ‘best’ fit out of the options may not provide the optimal fit for a user, resulting in discomfort and reduced milk collection efficacy. There is a need to provide a ‘one-size-fits-all’ breast shield which eliminates the need to manufacture and package multiple different breast shields per breast pump. Further, there is a need to ensure such a breast shield can provide a good fit to all users with any nipple size.

Additionally, known breast pump systems comprise separate diaphragm components and breast shield components. This, along with the provision of multiple breast shields, increases the product size and manufacturing demands. There is a need to simplify the components of a breast pump system.

### SUMMARY

There may be provided a breast shield for a breast pump comprising a flexible diaphragm comprising a nipple tunnel for receiving a user's nipple. The breast shield may be configured to form a seal with a user's breast and is configured such that when an internal negative pressure is applied to internal walls of the nipple tunnel the nipple tunnel contracts to a minimum aperture which conforms to the shape of the user's nipple.

The nipple tunnel may comprise external walls, wherein when an external negative pressure is applied to the external walls, the nipple tunnel expands radially up to a maximum aperture.

The nipple tunnel may comprise a plurality of expandable lobes which extend radially outward from a central longitudinal axis of the breast shield.

The nipple tunnel may comprise three lobes that dilate between the maximum aperture and the minimum aperture.

The three lobes may each extend radially from a point offset from a central longitudinal axis of the breast shield.

The maximum aperture may comprise a convex circular triangle.

The nipple tunnel may comprise a single lobe, the single lobe comprising at least one expandable area.

The single lobe may comprise a rounded triangular prism shape, with three expanding areas.

The nipple tunnel may comprise four lobes that dilate between the maximum aperture and the minimum aperture.

The nipple tunnel may comprise a central circular or cylindrical portion, and two T-shaped lobes.

The nipple tunnel may approach a circular aperture at maximum aperture.

The flexible diaphragm may be formed as a single piece, optionally by an injection moulded process or a compression moulded process, and optionally or additionally wherein the flexible diaphragm is formed of silicone.

The breast shield may comprise a non-return valve at one end of the nipple tunnel. Any embodiment of breast shield as described herein may comprise a non-return valve at one end of the nipple tunnel.

The breast shield may comprise a breast flange portion. Any embodiment of breast shield as described herein may comprise a breast flange portion.

The breast shield may be optically transparent, optically translucent or optically opaque. Any embodiment of breast shield as described herein may be optically transparent, optically translucent or optically opaque.

The breast shield may comprise sizing rings. Any embodiment of breast shield as described herein may comprise sizing rings.

There may be provided a breast pump comprising a pumping system and a breast shield. The pumping system may be configured to apply an internal negative pressure to the internal walls of the nipple tunnel.

The application of the internal negative pressure may form a vacuum seal between the breast shield and a user's breast.

The pumping system may be further configured to apply an external negative pressure to external walls of the nipple tunnel.

The breast pump may comprise an outer frame configured to define a region surrounding the nipple tunnel.

The external negative pressure may be greater in magnitude than the internal negative pressure.

A level of suction between the breast shield and the user's breast may be highest when both the internal negative pressure and the external negative pressure are applied to the nipple tunnel.

The pumping system may apply the external negative pressure to the external walls of the nipple tunnel periodically during a pumping cycle, causing expandable lobes of the nipple tunnel to periodically expand and contract between the minimum aperture and the maximum aperture.

The breast shield may be removable from the breast pump. Any embodiment of breast shield described herein may be removable from the breast pump.

The breast shield according to any embodiment as described herein may be removed and attached to the breast pump using a joint mechanism arranged on the outer frame, optionally wherein the joint mechanism is configured for a one press action optionally comprising audible and/or haptic feedback to indicate attachment and/or removal.

The breast pump may be configured as a self-contained, in-bra wearable device. Any embodiment of breast pump described herein may be configured as a self-contained, in-bra wearable device.

There may be provided a breast shield for a breast pump comprising a flexible diaphragm comprising a nipple tunnel for receiving a user's nipple, and an outer frame comprising a first extension configured to define a first region for application of negative pressure to external walls of the nipple tunnel. The breast shield may be configured to form a seal with a user's breast and is configured such that when an external negative pressure is applied to the external walls of the nipple tunnel in the first region, the nipple tunnel expands in at least a longitudinal direction.

When an internal negative pressure is applied to internal walls of the nipple tunnel the nipple tunnel may contract to a minimum aperture which conforms to the shape of the user's nipple.

The outer frame may comprise a second extension configured to define a second region for application of positive pressure to the external walls of the nipple tunnel.

When an external positive pressure is applied to the external walls of the nipple tunnel in a second region, the nipple tunnel may contract to a minimum aperture which conforms to the shape of the user's nipple.

The nipple tunnel aperture may be bi-stable, such that as the breast shield is pushed onto the user's breast, a diameter of the nipple tunnel aperture decreases in size from a first stable aperture to a second stable aperture. The second stable aperture may be smaller in diameter than the first stable aperture. Any embodiment of breast shield described herein may comprise a bi-stable nipple tunnel aperture.

There may be provided a breast pump comprising a pumping system and a breast shield. The pumping system may be configured to apply an external negative pressure to the external walls of the nipple tunnel in the first region.

The pumping system may be further configured to apply an internal negative pressure to internal walls of the nipple tunnel.

The pumping system may be further configured to apply an external positive pressure to the external walls of the nipple tunnel in the second region.

The pumping system may apply the external negative pressure to the external walls of the nipple tunnel in the first region periodically during a pumping cycle, causing the nipple tunnel to periodically expand and contract.

There may be provided a breast shield for a breast pump comprising a diaphragm comprising a nipple tunnel for receiving a user's nipple, a breast flange portion and an outer frame configured to define a region surrounding the nipple tunnel. The nipple tunnel may comprise a single lobe and at least one inflation pocket configured to receive positive pressure. Application of positive pressure to the at least one inflation pocket may cause expansion of the lobe in at least one of a longitudinal, a radial and/or a lateral direction.

The end of the lobe furthest from the breast flange portion may be coupled to the outer frame.

The at least one inflation pocket may comprise a single inflation pocket extending around the circumference of the lobe at the end of the lobe closest to the breast flange portion, the single inflation pocket optionally comprising a torus shape.

The at least one inflation pocket may comprise a plurality of inflation pockets positioned at various points around the circumference of the lobe at the end of the lobe closest to the breast flange portion.

There may be provided a breast pump comprising a pumping system and a breast shield. The pumping system may be configured to apply a positive pressure to the at least one inflation pocket.

The pumping system may apply the positive pressure to the at least one inflation pocket periodically during a pumping cycle, causing the lobe to periodically expand and contract.

### BRIEF DESCRIPTION OF THE DRAWINGS

Specific embodiments are described below by way of example only and with reference to the accompanying drawings in which:

FIG. 1 shows a breast pump according to an embodiment of the invention;

FIGS. 2A-C show a breast shield for a breast pump according to an embodiment of the invention;

FIG. 3A shows a breast shield according to an embodiment of the invention, comprising a nipple tunnel having three lobes and in a ‘collapsed’position;

FIG. 3B shows a breast shield according to an embodiment of the invention, comprising a nipple tunnel having three lobes and in an ‘expanded’position;

FIGS. 4A-C show a breast shield and outer frame according to an embodiment of the invention;

FIGS. 5A and 5B show a breast shield according to an embodiment of the invention, comprising a nipple tunnel having three lobes;

FIGS. 6A-C show a breast shield according to an embodiment of the invention, comprising a nipple tunnel having four lobes;

FIGS. 7A-C show a breast shield according to an embodiment of the invention;

FIGS. 8A-C show a breast shield according to an embodiment of the invention, comprising a single lobe and two expanding areas;

FIGS. 9A and 9B show a breast shield according to an embodiment of the invention, comprising a single lobe and three expanding areas;

FIGS. 10A and 10B show a breast shield according to an embodiment of the invention, comprising a non-return valve;

FIGS. 11A and 11B show a nipple tunnel of a breast shield according to an embodiment of the invention, comprising inflation pockets.

FIGS. 12A-C show a breast shield according to an embodiment of the invention, comprising a passively collapsible nipple tunnel;

FIGS. 13A and 13B show a breast shield according to an embodiment of the invention, comprising an actively collapsible nipple tunnel;

FIGS. 14A and 14B show a breast shield according to an embodiment of the invention, comprising a bi-stable nipple tunnel aperture;

FIGS. 15A and 15B show a breast shield according to an embodiment of the invention;

FIGS. 16A and 16B show a breast shield according to an embodiment of the invention;

FIG. 17 shows a breast shield according to an embodiment of the invention, comprising sizing rings.

FIG. 18 shows a breast shield according to an embodiment of the invention, comprising a stiffening element.

### DETAILED DESCRIPTION OF THE EMBODIMENTS

### General configuration

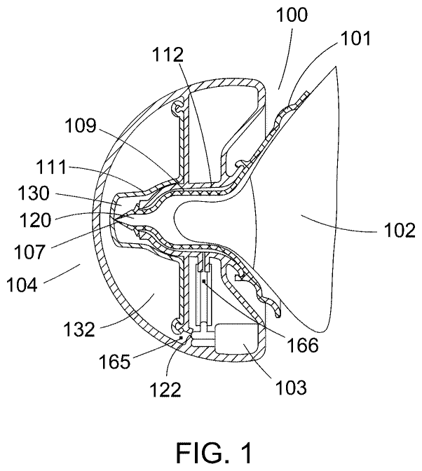

A breast pump 100 according to an embodiment of the claimed invention is shown in FIG. 1.

### Breast Pump

The breast pump may comprise a pumping system and a breast shield. The pumping system may comprise an air pump, and may be configured to generate and/or apply negative air pressure. The breast pump 100 is suitable for expressing human breast milk. The assembled breast pump 100 system may comprise a housing 110 shaped to substantially fit inside a bra. The housing may be designed to enclose all the components of the breast pump 100. The housing may be shaped to discreetly fit underneath a user's clothing or to be worn inside a bra. The housing may comprise a breast shield 101 for fitting to a user's breast 102, at least one air-pump 103 for generating a vacuum and a milk container (not shown). The housing may also comprise a rechargeable battery and control electronics (not shown). The breast pump 100 may be configured as a self-contained, in-bra wearable device. The breast shield 101 and milk container 104 may also be configured as being in-bra wearable.

The only parts of the system that come into contact with milk in normal use are the breast shield 101 and the milk container 104 and any portion of the breast pump 100 which connects the breast shield 101 and the milk container 104, such as a nipple tunnel 109 and non-return valve 107. In some embodiments, milk only flows along a milk path through the breast shield 101 and then directly into the milk container (not shown). Milk does not contact the housing in such embodiments, for maximum hygiene and ease of cleaning.

The breast shield 101 and the milk container 104 may be removable from or attachable to the housing in normal use or during normal dis-assembly. Other parts that are user-removable in normal use or during normal dis-assembly are attached to either the breast shield 101 or the milk container. The breast shield 101 and milk container may be removed from or attached to the housing, for example, using a one click or one press action or a push button or any other release mechanism such as magnetic or screw attachments. Audible and/or haptic feedback may confirm that the pump is properly assembled.

The modularity of the breast pump 100 may allow for easy assembly, disassembly and replacement of different parts such as the breast shield 101 and milk collection container. This may also allow for different parts of the pump to be easily washed and/or sterilised. The breast shield 101 and container assembly, both of which are in contact with milk during pumping, may therefore be efficiently and easily cleaned; these are the only two items that need to be cleaned due to contact with breast milk; in particular, the housing does not need to be cleaned.

### Breast Shield

The breast shield 101 may comprise a flexible diaphragm which comprises a nipple tunnel 109 for receiving a nipple. The breast shield may be configured to form a seal with a user's breast. The breast shield may also comprise a breast flange portion 108 for fitting to the user's breast 102. The breast flange 108 contacts the user's breast 102 and seals the breast shield 101 to the surface of the user's breast 102. The flexible diaphragm may be a single piece diaphragm.

In some embodiments, the flexible diaphragm may be considered to comprise the nipple tunnel. That is to say, the nipple tunnel may be a part of the flexible diaphragm such that the nipple tunnel may be considered to be a flexible nipple tunnel. Any reference herein to manipulation, shaping, expansion, contraction and the like of the nipple tunnel may alternatively or additionally be considered to be manipulation, shaping, expansion, contraction and the like of the flexible diaphragm. Application of pressure to the nipple tunnel may be considered to be application of pressure to the flexible diaphragm. Any reference herein to the shape and/or structure and the like of the nipple tunnel may be considered to be the shape and/or structure and the like of the flexible diaphragm.

In alternative embodiments, the nipple tunnel may comprise a flexible portion and a non-flexible portion. In such embodiments, the breast shield may comprise a nipple tunnel comprising a flexible portion and a non-flexible portion. The breast shield may further comprise a flexible diaphragm comprising the flexible portion of the nipple tunnel. In embodiments wherein the nipple tunnel comprises a flexible portion, any reference herein to manipulation, structure and shaping, expansion, contraction, application of pressure and the like of the nipple tunnel may be considered to be manipulation, shaping, expansion, contraction, application of pressure and the like of the flexible portion of the nipple tunnel, and/or of the flexible diaphragm. Application of pressure to the nipple tunnel may be considered to be application of pressure to the flexible portion of the nipple tunnel, and/or of the flexible diaphragm. Any reference herein to the shape and/or structure and the like of the nipple tunnel may be considered to be the shape and/or structure and the like of the flexible portion of the nipple tunnel, and/or of the flexible diaphragm.

Any embodiment of breast shield and/or nipple tunnel as described herein may be compatible with a nipple tunnel comprising a flexible portion and a non-flexible portion.

It may be desirable to reduce excess air volume in the system to enable performance gains in the maximum pressure generated inside the nipple tunnel 109, therefore increasing the efficacy of milk production.

The nipple tunnel 109 may comprise internal walls. The nipple tunnel may also comprise external walls. The breast shield 101 may optionally be designed to be flexible so that it may collapse and expand when exposed to different pressures generated by the air pump 103 on the internal and external walls of the nipple tunnel.

In particular, the nipple tunnel 109 may comprise a plurality of expandable lobes which enable the nipple tunnel to expand and contract in a radial direction between a minimum volume and a maximum volume during a pumping cycle of the breast pump 100. The minimum volume corresponds to a minimum diameter of nipple tunnel, the diameter being taken perpendicular to the central axis of the nipple tunnel. The maximum volume corresponds to a maximum diameter of the nipple tunnel. Exemplar embodiments of the breast shield 101 comprising various exemplar configurations of the expandable lobes will be described in more detail with reference to subsequent figures.

The breast shield 101 according to an embodiment of the present invention may be configured to form a fluid-tight seal between the breast shield 101 and the user's breast. This seal may be maintained during use of the breast pump 100, and optionally for periods before and/or after pumping. This prevents leakage of fluids such as air and/or milk between and/or after pumping cycles. The seal may be a vacuum seal, and will be described in more detail in the following section with reference to a base level vacuum. Additionally, the seal between the breast flange portion 108 of the breast shield 101 and the user's breast ensures air is only pumped from the inside of the breast shield, i.e. the side of the shield configured to receive the breast, and not from the side of the breast shield 101 facing away from the breast shield.

The flexible diaphragm comprising the nipple tunnel may be made from a continuous piece of flexible material. The flexible material has specific properties, such as shore hardness and/or constant thickness. An example of such a material is silicone. The flexible diaphragm may be formed by an injection or compression moulding process. The flexible diaphragm may be formed as a single piece.

The parameters of the breast shield may be configured to enhance the overall performance and user experience. Parameters comprise, for example, shore hardness, overall geometry or size, thickness etc.

### Base Level Vacuum

When a nipple is placed inside the nipple tunnel 109, the breast shield 101 may be acted on by a vacuum applied on the internal walls of the nipple tunnel 109, i.e., an internal negative pressure is applied to the internal walls of the nipple tunnel. This internal negative pressure is referred to hereinafter as the base level vacuum. The breast pump 100 disclosed herein may be able to achieve a base level vacuum throughout a whole pumping cycle. This means that a minimum base level vacuum is produced to enable the breast shield 101 to maintain contact with the user's breast 102 at all times during the pumping process. In other words, the base level vacuum, i.e. internal negative pressure, may enable the breast shield 101 to maintain a fluid-tight vacuum seal between the shield and the user's breast at all times during the pumping process. Additionally or alternatively, the seal may be maintained by the base level vacuum before and/or after the pumping process. For example, the seal may be maintained before commencement of a pumping cycle once the breast pump 100 has been arranged on the user's breast. Additionally or alternatively, the seal may be maintained after finishing a pumping cycle until either the next pumping cycle is commenced or the breast pump 100 is to be removed from the user's breast. This prevents leakage of milk between and/or after pumping cycles.

The base level vacuum means that the pressure in the breast shield, and specifically the nipple tunnel 109, never reaches or rises above atmospheric pressure. Instead, a constant negative air pressure is achieved in the nipple tunnel 109 to maintain contact between the user's breast 102 and the breast shield 101. The base level vacuum may be described as causing a suction between the breast shield 101 and a user's breast. The term suction is used herein to reflect the use of negative pressure to maintain an active seal between the shield 101 and the user's breast.

When the internal negative pressure is applied to the internal walls of the nipple tunnel, the nipple tunnel contracts to a minimum aperture which conforms to the shape of the user's nipple. In other words, upon actuation of the base level vacuum, the nipple tunnel 109 is configured to contract, or collapse, and conform to the shape of a user's nipple, and maintain a constant suction between the nipple and the breast shield 101. The nipple tunnel 109, when contracted as such, conforms to a tight fit around the user's nipple. The base level vacuum provides a feeling of biomimicry, as though a child is latched on throughout the pumping process, for a user and also ensures a leak free experience by sealing the breast shield 101 to the user's breast 102.

The desired pressure of the base level vacuum may be individual to the user. It is a pressure determined by what the user perceives and/or requires to be strong enough to maintain a secure seal against the breast throughout the pumping session but without becoming uncomfortable. This provides a seal between the breast and the breast shield 101 at all times during the pumping cycle, thereby reducing the likelihood of milk leaking from the breast shield 101.

For example, the user can be given the option to choose a desired base level vacuum of either-25 mmHg,-50 mmHg or -75mmHg (relative to atmospheric pressure). This option can be displayed to a user via a graphical user interface on a digital application, or alternatively or additionally via a user interface on the pump 100. The choice of desired base level vacuum may provide a tunable ‘feeling’ to allow the user to find a setting that feels like a personal breast-feeding experience.

The base level vacuum may also be applied to expel all redundant air within the breast shield 101. This ensures that the nipple tunnel 109 and breast flange 108 sit as close to the nipple and breast as possible and maximises the possible amount of air volume on an external side of the flexible diaphragm (i.e. a side of the flexible diaphragm not receiving the user's breast) to make the best use of the air in the breast pump system. The volume of air inside the nipple tunnel is minimised, and so the volume of air that the base level vacuum must act on is minimised. The base level vacuum offers the additional benefits of sealing the device to the user's breast to offer a reassuring fit and tactile confirmation that the device is firmly in place. By ensuring the nipple tunnel 109 and breast flange 108 sit as close to/mould to the nipple/breast as possible, the breast shield 101 also fits to any size of nipple and supports the areola from being pulled into the breast shield.

When a nipple is placed inside the nipple tunnel 109, the pump 103 is actuated, which causes a flexible part of the breast shield, referred to herein as a flexible diaphragm to expand or contract radially, which in turn causes the nipple to be stimulated and/or to be drawn into the nipple tunnel. The diaphragm continues to expand or contract radially, causing milk to be drawn from the nipple. The milk is then collected into the nipple tunnel 109 and flows along a milk path to the collected in the milk container (not shown).

### Air Pump

The air pump 103 is a mechanical air pump 103 designed to either extract air from a breast pump 100 system or insert air into a breast pump 100 system. In embodiments shown herein, the air pump 103 may be configured for a first level of pumping to generate the base level vacuum. The air pump 103 may additionally or alternatively be configured for a second level of pumping to generate a pumping vacuum for milk expression. In some embodiments, the system comprises a first level of pumping to generate the base level vacuum, i.e. to generate an internal negative pressure to be applied to the internal walls of the nipple tunnel, and a second air pump level of pumping to generate a pumping vacuum for milk expression, i.e. an external negative pressure to be applied to the external walls of the nipple tunnel.

When configured to generate the base level vacuum, the air pump 103 is configured to draw air out of the breast pump 100 system and create a negative air pressure in the nipple tunnel 109, such that a negative air pressure acts on the internal walls of the nipple tunnel 109.

When configured to generate a pumping vacuum, for example during a pumping cycle, the air pump 103 is configured to draw air out of the breast pump 100 system and create a negative air pressure in a region surrounding the nipple tunnel 109, such that a negative air pressure acts on the external walls of the nipple tunnel 109. This external negative air pressure acting on the external walls of the nipple tunnel 109 may be more negative than the internal negative air pressure acting on the internal walls of the nipple tunnel 109, such that the net effect is a negative air pressure acting on the external walls of the flexible diaphragm. This pulls the lobes of the nipple tunnel radially outwards, increasing the volume in the nipple tunnel. The increased volume in the nipple tunnel, along with the seal between the user's breast and the reduced air volume in the tunnel caused by the base level vacuum, creates a high suction level in the nipple tunnel. This high suction in the nipple tunnel assists in drawing milk out of the user's breast and into the nipple tunnel.

In other words, a negative air pressure differential is created between the air pump 103 and the inside of the nipple tunnel 109. This net negative pressure is thereby applied to the nipple, drawing milk from the breast which is collected inside the milk container 104.

Since the net effect of the pumping vacuum and the base level vacuum is a net negative pressure differential applied to the nipple, the base level vacuum can be maintained during a pumping cycle such that suction between the breast shield 101 and the user's breast can be maintained whilst the air pump 103 applies varying pressures to the outside of the flexible diaphragm for milk collection.

A negative air pressure differential is defined as any pressure below that of the surrounding air environment. In other words, a negative air pressure differential is a pressure lower than the system of the breast pump 100 when the air pump 103 is not in use. A typical air pressure, or atmospheric pressure, of a standard environment is 760 mmHg, therefore a negative air pressure may be defined as any pressure lower than 760 mmHg. The term negative pressure as used herein may be considered to refer to or be a negative air pressure differential.

Optionally, the air pump 103 may also be configured to generate and/or apply a positive pressure. This positive pressure may be applied to the internal walls of the nipple tunnel. For example, a positive air pressure may be used to assist with emptying or evacuating of elements of the breast pump 100. In particular, a positive air pressure can be used to expel milk from the milk bottle. Positive air pressure may be applied to the external walls of the nipple tunnel. Positive pressure applied to the external walls of the nipple tunnel may be used to deform the nipple tunnel to conform to a user's nipple.

The air pump 103 may be a rotary diaphragm pump. A rotary diaphragm pump is a positive displacement pump that uses a combination of the reciprocating action of a flexible diaphragm (e.g. made from silicon rubber or thermoplastic) and suitable valves on either side of the diaphragm to pump a fluid (in some embodiments of the present disclosure, the rotary diaphragm pump pumps air). A rotary air pump provides a cost effective and easy way to reach desired pressures. In this case, the air pumping subsystem may either be configured as an open loop or closed loop pumping subsystem. The rotary diaphragm pump used may be a standard rotary diaphragm pump as is known for use in breast pump 100.

Alternatively, the air pump 103 may be a piezoelectric pump. Piezoelectric air pumps (or piezo pumps), operate silently (e.g. outside the range of human hearing) and with minimal vibrations. Due to their low noise, strength and compact size, piezoelectric pumps are ideally suited to the embodiment of a small, wearable breast pump. However, piezo pumps generate higher heat as compared to, for example, rotary diaphragm pumps. Reducing the air volume in the system improves the efficiency of the pump, and the pump therefore generates less heat. When a piezoelectric pump is used, the air pumping subsystem may also either be configured as an open loop or closed loop pumping subsystem.

Other possible types of pumps are also feasibly usable in the disclosure. For example, a peristaltic or vein pump could also be used.

In some embodiments, the pump 103 may be housed within the in-bra breast pump 101. However, the pump may optionally be housed separately and connected to the in-bra architecture by simple tubing. In any embodiment of the pump 103 as described herein, the pump may be considered to be part of a pumping system. In other words, the breast pump 100 may comprise a pumping system. The pumping system may comprise the pump 103.

The wearable breast pump 100 is configured to operate quietly in normal use. A cavity containing the air pumping subsystem (comprising the air pump 103) may be sealed and comprise other noise reduction technology so as to further attenuate sound.

The air pump 103 is configured to pump at a wide range of different levels of intensity. A first level of pumping is provided to generate the base level vacuum inside the nipple tunnel 109. A second level of pumping is provided to generate a pumping vacuum to stimulate the breast tissue and initiate milk expression from the breast. The second level of pumping is more intense than the first level. This is because a negative air pressure of greater magnitude must be generated for when the air pump 103 is expressing milk from the user's breast 102, compared to when only the base level vacuum desired pressure is required.

In one embodiment, the first level of pumping is configured to produce a negative air pressure of from-15 to-70 mmHg (relative to atmospheric pressure) inside the nipple tunnel 109. In another embodiment, the first level of pumping Is configured to produce a negative air pressure of from-30 to-60 mmHg (relative to atmospheric pressure) inside the nipple tunnel 109. In yet another embodiment, the first level of pumping is configured to produce a negative air pressure of-50mmHg, plus or minus 10% (relative to atmospheric pressure) inside the nipple tunnel 109. The above pumping pressures are disclosed as merely examples, and the skilled person would understand that other ranges may be possible. The first level of pumping may also be determined by a user's preferences and input, via a connected device or additionally or alternatively via a user interface on the pump 100, to the breast pump.

In one embodiment, second level of pumping is configured to produce a negative air pressure of from-150 to-300 mmHg (relative to atmospheric pressure) inside the nipple tunnel 109. In another embodiment, the second level of pumping may be configured to produce a negative air pressure of from-200 to-280 mmHg (relative to atmospheric pressure) inside the nipple tunnel 109. In yet another embodiment, the second level of pumping may be configured to produce a negative air pressure of from-230 to-280 mmHg (relative to atmospheric pressure) inside the nipple tunnel 109. The above pumping pressures are disclosed as merely examples, and the skilled person would understand that other feasible ranges are possible. The second level of pumping may also be determined by a user's preferences and input via a connected device to the breast pump, or alternatively or additionally via a user interface on the pump 100. The user may choose, for example, a smaller magnitude of second pumping level to improve comfort. Alternatively, the user may choose, for example, a greater magnitude of second pumping level to improve milk expression efficacy.

The desired second level of pumping may be individual to the user. The second level of pumping is greater in magnitude than the first level of pumping. As will be understood by the skilled person, any second level of pumping greater in magnitude than the first level of pumping may be selected in order to stimulate milk expression from the nipple. The second level of pressure may be determined by what the user perceives and/or requires to be strong enough to stimulate milk expression throughout the pumping session but without becoming uncomfortable. The level of suction between the breast shield and the user's breast may be highest when both the internal negative pressure and the external negative pressure are applied to the nipple tunnel.

For example, the user can be given the option to choose a desired second level of pumping configured to produce a negative air pressure of any magnitude greater than that of the first level of pumping, up to a maximum of-300mmHg (relative to atmospheric pressure). This option can be displayed to a user via a graphical user interface on a digital application, or alternatively or additionally via a user interface on the pump 100. The choice of desired pumping vacuum may provide a tuneable ‘feeling’ to allow the user to find a setting that feels like a personal breast-feeding experience.

A pumping cycle may comprise a plurality of actuations of the air pump 103, such that the pumping cycle comprises a plurality of oscillations between the first level of pumping and the second level of pumping. When the internal negative air pressure acts alone on the internal walls of the nipple tunnel, i.e. only the base level vacuum is present as a result of the air pump 103 operating at the first level of pumping, the expandable lobes of the nipple tunnel are arranged in a collapsed position with a minimum aperture. When the external negative air pressure acts on the external walls of the nipple tunnel in addition to the internal negative air pressure, i.e. both the base level vacuum and pumping vacuum are present as a result of the air pump 103 operating at a second level of pumping, the expandable lobes of the nipple tunnel are arranged in an expanded position with a maximum aperture. The external negative pressure may be greater in magnitude than the internal negative pressure. Consequently, during a pumping cycle, the nipple tunnel 109 expands and contracts radially between the minimum aperture and the maximum aperture. The pumping system may apply the external negative pressure to the external walls of the nipple tunnel periodically during a pumping cycle, causing the nipple tunnel to periodically expand and contract between the minimum aperture and the maximum aperture.

In general, for breast pump 100 systems comprising any embodiment of breast shield as described herein, the pumping system of the breast pump may apply external negative pressure to the external walls of the nipple tunnel periodically during a pumping cycle, causing the nipple tunnel to periodically expand and contract.

The negative pressure produced by the second level of pumping, i.e. the pumping vacuum, from the pump 103 acts to cause radial expansion and contraction of nipple tunnel 109, which will be described in more detail as follows.

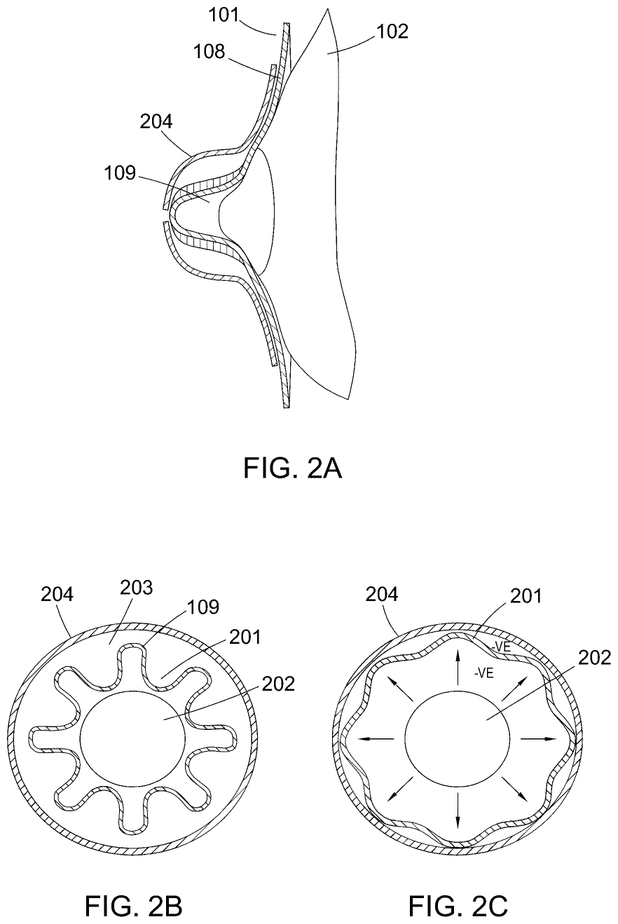

FIG. 2A shows a breast shield for a breast pump according to an embodiment of the present invention. The breast shield 101 is configured for receiving a user's breast 102. The breast shield 101 comprises a breast flange portion 108, and a flexible fluted nipple tunnel 109 that also acts as a diaphragm. The flange portion 108 is configured to lie in contact with, and in some embodiments flush with, the surface of the user's breast. The nipple tunnel 109 receives the user's nipple, and is connected to the breast flange portion 108.

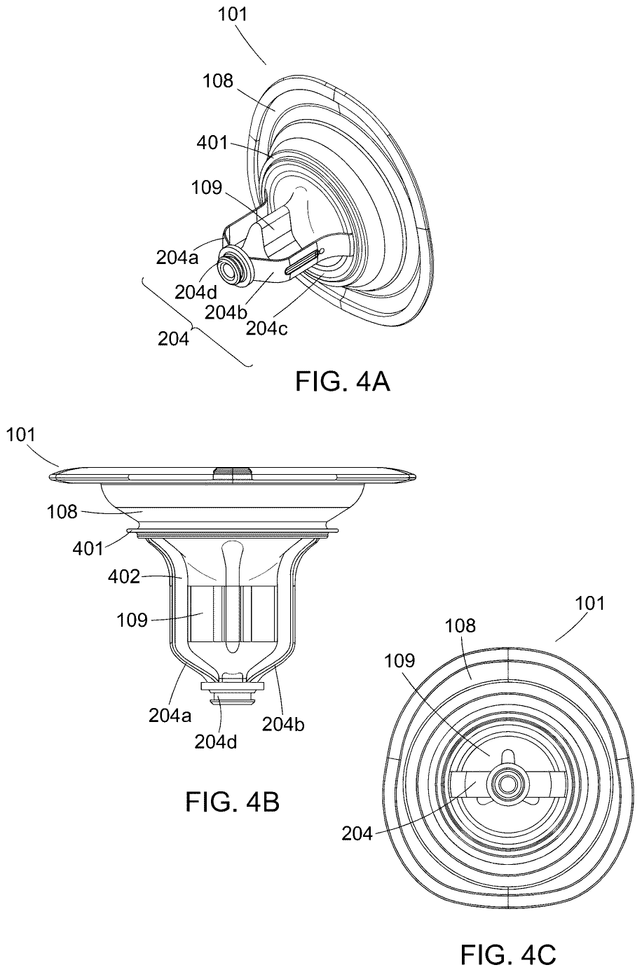

The flexible diaphragm or membrane comprises the nipple tunnel 109, that is configured to deform or expand as pressure is reduced or increased in the region(s) between the flexible nipple tunnel and an outer frame 204. The outer frame 204 may be a rigid outer frame, which may comprise a rigid material. The term outer frame as used herein may be considered to be an outer tunnel. The frame will be described further in reference to FIG. 4.

The outer frame 204 is not necessarily the outermost part of the device. In some examples, the outer frame 204 could be internal to another frame or another part of the device. For example, the outer frame 204 could be internally overmoulded as part the device. The outer frame 204 may be referred to as a frame 204.

The nipple tunnel 109 may comprise a plurality of expandable lobes, or folds or pleats extending radially from a center or opening of the nipple tunnel 109. In particular, the lobes, or folds or pleats, may extend radially outward from a central longitudinal axis of the breast shield. The nipple tunnel 109 comprises at least one lobe, fold or pleat extending radially from a center or opening of the nipple tunnel. As shown in FIGS. 2B and 2C, the nipple tunnel 109 may comprise a plurality of lobes, or folds or pleats extending radially from a center or opening of the nipple tunnel. The embodiment shown in FIG. 2 comprises eight lobes 201. Optionally, the nipple tunnel 109 may have a single lobe or region that deforms or expands as pressure is reduced. Optionally, the nipple tunnel 109 may have two separate lobes or regions that each deform or expand as pressure is reduced. Optionally, the breast shield may have three lobes or regions that each deform or expand as pressure is reduced. Optionally, the breast shield may have four or more lobes or regions that each deform or expand as pressure is reduced. The different lobes may have different parameters, such as different amplitudes or thicknesses, and/or their lengths of extension along the nipple tunnel may vary. In other examples, the lobes are uniform in their dimensions.

When a nipple is placed inside the nipple tunnel 109, the breast shield 101 is acted on by the base level vacuum where air is pumped out from the inside (i.e. breast-facing side) of the breast shield. Air is pumped out from the sealed chamber formed between the breast and the breast shield. Upon actuation of the base level vacuum, i.e., when an internal negative pressure is applied to the internal walls of the nipple tunnel, the expandable lobes of the nipple tunnel 109 are configured to contract to a minimum aperture which conforms to the shape of the user's nipple. The shield contracts under the base level vacuum and maintains a constant suction between the nipple and the breast shield 101. This is shown in FIG. 2B, wherein the lobes 201 are in a ‘collapsed’ or ‘relaxed’ position, in which the nipple tunnel 109 is collapsed onto an inserted nipple 202, conforming to a tight fit around the nipple 202.

The lobe(s) of the nipple tunnel may comprise internal walls and external walls. Any pressure, positive or negative, referred to herein as applied to the internal or external walls of the nipple tunnel may be considered to be applied at least in part to the internal or external walls respectively of the at least one lobe. The internal walls of the nipple tunnel may comprise the internal walls of the lobe(s). The external walls of the nipple tunnel may comprise the external walls of the lobe(s).

When collapsed, only a very small volume of air is present inside the nipple tunnel. This enables performance gains in maximum pressure and cycle time. The conforming of the nipple tunnel to the shape of an inserted nipple may be automatic, in that it happens as a result of a base level vacuum being created in the nipple tunnel, i.e. as a result of the application of negative pressure to the internal walls of the nipple tunnel, without the need for the user to choose or adjust the size of the breast shield to the nipple.

Once the breast shield is securely placed on the user's breast and under constant suction provided by the base level vacuum a separate, pumping vacuum is applied. To achieve the pumping vacuum, the pump 103 may be actuated and pump air from the outside of the breast shield (i.e. the side facing away from the breast). Air is pumped from the region between the nipple tunnel and the outer frame 204. This region is sealed in use, as described in more detail with reference to FIG. 4. When air is pumped from the region between the breast shield 101 and outer frame 204, a net negative air pressure differential is created between the air pump 103 and inside the nipple tunnel 109. This causes the expandable lobes 201 of the nipple tunnel 109 to expand in a radial direction, in turn causing radial expansion of the user's nipple, drawing milk into the nipple tunnel 109. In other words, an external negative pressure is applied to the external walls of the nipple tunnel 109. When the external negative pressure is applied, the expandable lobes of the nipple tunnel expand radially up to a maximum aperture.

As the pressure is decreased, i.e. becomes more negative, in the region 203 between the nipple tunnel 109 and the outer frame 204, i.e. when the pressure acting on the external walls of the nipple tunnel becomes more negative, the lobes deform or expand to the ‘expanded’ configuration shown in FIG. 2C. FIG. 2C shows the position of the lobes when they are fully, or nearly fully, dilated in response to the application of negative pressure. The nipple opening, as shown in FIG. 2C, deforms continuously from a minimum aperture to a maximum aperture. The maximum aperture may be substantially more circular in shape than the minimum aperture. Since a base level vacuum is maintained within the nipple tunnel, as the breast shield expands radially to the expanded configuration, the nipple moves with the shape of the lobes of the nipple shield. This mimics sucking from an infant mouth. Because the shape or configuration of the nipple tunnel 109 is dynamic, the shape of the nipple tunnel is able to always be only as big as it needs to be, thereby reducing the air volume inside the nipple tunnel. Advantageously, the opening of the nipple tunnel at the interface with the breast can be increased when placing the nipple inside the nipple tunnel, and once the nipple is correctly placed inside the nipple tunnel, the opening at the interface with the breast can collapse to the desired size around the breast or nipple area.

The dilation, or deformation or expansion, and/or contraction of the expandable lobes may be automatic, in that the expansion and/or contraction happens as a result of the negative air pressure differential between the air pump 103 and the nipple tunnel 109, without the need for the user to manually manipulate the breast shield and/or manually actuate the air pump. The expansion of the nipple tunnel and/or the expandable lobes may be automatic in that the expansion happens as a result of the external negative air pressure applied to the external walls, without need for manual actuation of the pump by the user. The contraction of the nipple tunnel and/or the expandable lobes may be automatic in that the contraction happens as a result of the external negative air pressure no longer being applied to the external walls, without need for manual actuation of the pump by the user. The contraction of the nipple tunnel and/or expandable lobes may also happen as a result of the internal negative pressure applied to the internal walls.

The outer frame 204 may be a rigid outer frame or tunnel, which may comprise a frame made of a rigid material. The flexible diaphragm comprising the nipple tunnel may be made from a continuous piece of flexible material. The flexible material has specific properties, such as shore hardness and/or constant thickness, selected to provide high levels of suction during use of the breast pump. An example of such a material is silicone. The flexible diaphragm may be formed by an injection or compression moulding process.

The pumping system applies the external negative pressure to the external walls of the nipple tunnel periodically during a pumping cycle. This causes expandable lobes of the nipple tunnel to periodically expand and contract between the minimum aperture and the maximum aperture. The deformation or expansion of the lobes in response to reduced pressure surrounding the nipple tunnel, i.e. applied to the external walls of the nipple tunnel, can be achieved with several lobe arrangements and shapes, as will be described with reference to the following embodiments. The following embodiments are disclosed as merely examples, and the skilled person would understand that other feasible arrangements are possible.

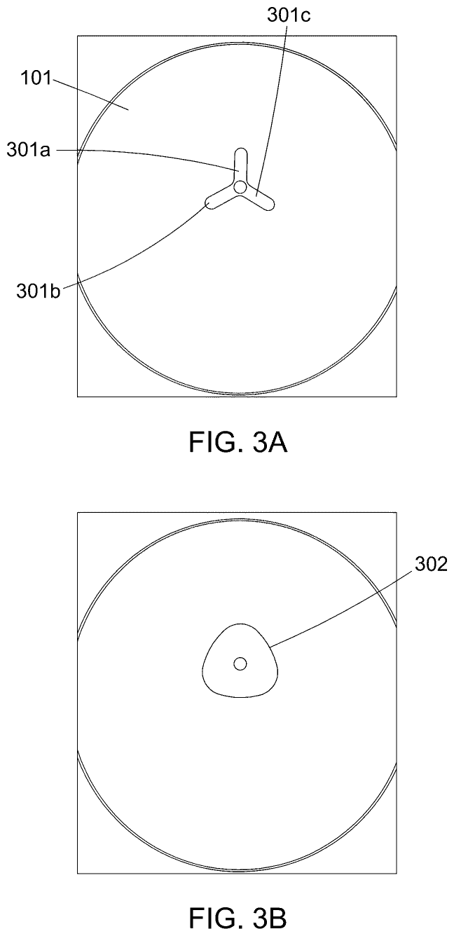

FIG. 3 shows an embodiment of a breast shield according to the present invention, as viewed from the ‘inside’; the side which, in use, is configured to receive the user's breast.

FIG. 3A shows the breast shield 101 in the ‘collapsed’ or ‘relaxed’ position. The embodiment of the breast shield shown in FIG. 3 comprises a single piece, flexible diaphragm comprising a nipple tunnel. The nipple tunnel is configured to comprise three lobes 301a, 301b, 301c which are configured to expand and contract between a maximum and minimum aperture. The lobes 301a, 301b, 301c extend radially from a center of the nipple tunnel 109, and may be spaced symmetrically about the center. The center may be considered to be a central longitudinal axis of the breast shield 101. FIG. 3A shows the lobes at or near the minimum aperture; FIG. 3B shows the lobes at or near the maximum aperture. Dilation of the lobes from minimum to maximum aperture in a radial direction is in response to the application of negative pressure to the outside of the flexible diaphragm, in particular to the external walls of the nipple tunnel. The opening 302 to the nipple tunnel approaches a maximum aperture when the lobes expand. The maximum aperture may comprise a circle. Alternatively, the maximum aperture may comprise a convex circular triangle. In particular, the convex circular triangle may approximately form a Reuleaux triangle. The opening to the nipple tunnel deforms continuously from a minimum aperture to a maximum aperture. The maximum aperture may be substantially more circular in shape than the minimum aperture. Therefore, the nipple tunnel approaches a circular aperture at maximum aperture. This is due, in part, to the natural consequence of having three or more lobes made from a continuous piece of flexible membrane.

The different lobes may each be substantially similar in shape, as shown in FIG. 3. Alternatively, the different lobes may have different parameters, such as different amplitudes or thicknesses, and their lengths of longitudinal extension along the nipple tunnel may vary.

FIG. 4 shows an embodiment of the breast shield with the outer frame according to the present invention. FIG. 4A shows a perspective view of the breast shield and outer frame, FIG. 4B shows a ‘top down’ view of the breast shield and outer frame, FIG. 4C shows an ‘end-on’ view of the breast shield and outer frame as viewed along a central longitudinal axis of the breast shield.

The outer frame 204 may comprise two or more support members 204a, 204b extending from a central support ring 204c. The support ring 204c may be configured to couple the frame 204 to the breast shield 101 at or near the base of the nipple tunnel 109, i.e., at or near the interface between the nipple tunnel 109 and breast flange 108. The outer frame 204 may be removably attached to the breast shield 101, or may be joined to the breast shield 101 such that the breast shield 101 and outer frame 204 form a single piece.

The breast shield may be removed and attached to the breast pump 100 system using a joint mechanism arranged on the outer frame. The two or more support members 204a, 204b may meet at the end of the nipple tunnel 109 furthest from the breast flange 108, optionally forming a joint member 204d configured for connection and disconnection of the outer frame 204 and the breast shield 101 to the rest of the breast pump 100 system. The joint member 204d may be circular or cylindrical in shape, and optionally is configured for easy assembly with the breast pump 100 system. In particular, the joint mechanism may be configured for a one click or one press action or a push button or any other release mechanism such as magnetic or screw attachments. Audible and/or haptic feedback may confirm that the frame 204 is properly attached and the pump 100 is properly assembled.

In one exemplar embodiment the frame 204 may comprise a single piece, separate from the breast shield 101, and easily assembled with and/or removable from the breast shield 101. In alternative embodiments, the frame 204 may joined to the breast shield 101 such that the breast shield 101 and the frame 204 form a single, removable component of the breast pump 100 system. Both embodiments provide easy assembly and/or cleaning of the breast shield 101 and/or frame 204.

The frame 204 may define an outer boundary of a region 402 surrounding the nipple tunnel in which negative pressure may be generated to provide a negative pressure acting on the external walls of the nipple tunnel 109.

The breast shield 101 may comprise a sealing extension 401. This is best visualised in FIG. 4B. The sealing extension 401 may be substantially ring-shaped, extending around the circumference of the breast shield and may be positioned at or near the base of the nipple tunnel 109, i.e., at or near the interface of the nipple tunnel 109 and the breast flange 108. The sealing extension 401 may be configured to seal the region 402 surrounding the nipple tunnel 109 when the breast shield 101 and frame 204 are assembled with a breast pump 100 system, forming fluid-sealed region 402 around the nipple tunnel defined by the frame 204 and the sealing extension 401. Negative pressure may be applied to the external walls of the nipple tunnel 109 by e.g. pump 103 providing a pumping vacuum to the fluid (i.e. air) in the region 402. The support ring 204c (not shown in FIG. 4B) may be configured to couple the frame 204 to the breast shield 101 at or near the sealing extension 401, this can be visualised in FIGS. 4A and 4C. The joint member 204d may also be configured to provide a fluid-tight seal when the frame 204 is attached and assembled with the breast pump 100 system.

The nipple tunnel 109 of the breast shield 101 shown in FIGS. 4A-C comprises three expandable lobes. The frame 204 may be compatible for coupling to and/or for use with breast shields comprising any other configuration of nipple tunnel as described herein. The compatibility should not be seen as limited thereto, however, and further configurations of nipple tunnel may also be suitable as understood by the skilled person.

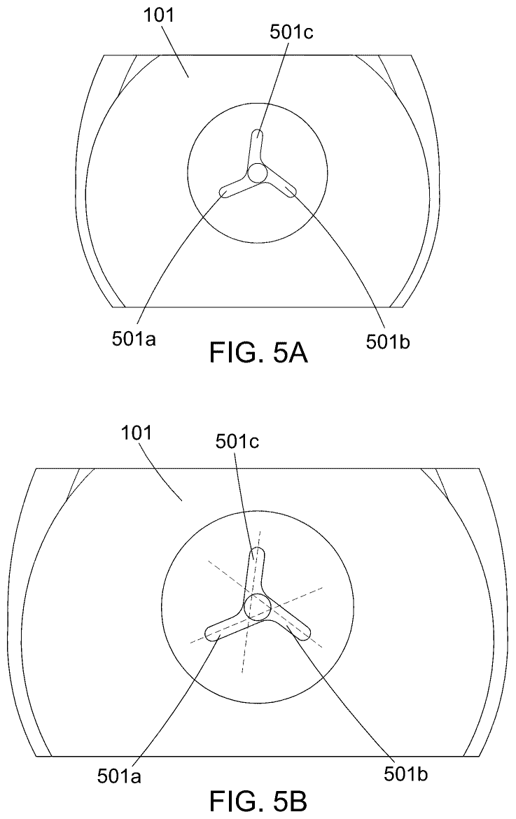

FIG. 5 shows an embodiment of the breast shield according to the present invention. FIGS. 5A and 5B show the breast shield at or near the collapsed position. Similar to the embodiment described with reference to FIG. 3, the embodiment shown in FIG. 5 comprises a nipple tunnel configured to comprise three separate lobes 501a, 501b, 501c. The lobes of the embodiment shown in FIG. 3 extend radially from a center of the nipple tunnel, and are spaced symmetrically about the center. The center may be considered to be a central longitudinal axis of the breast shield, which intersects the nipple tunnel at a central point. This may be the case for any embodiment of breast shield described herein.

The lobes in the embodiment shown in FIG. 5, however, extend radially from a point offset from the center of the nipple tunnel. In other words, in the off-center trilobe configuration shown in FIG. 5 the lobes radiate around the center, instead of radiating from the center of the nipple tunnel. Each lobe may extend from a different point respectively. This can be seen in FIG. 5B, with the dashed lines indicating the offset of each lobe. The points from which each lobe extends may form a triangle approximately centered on the center of the nipple tunnel. That is, the nipple tunnel comprises a plurality of expandable lobes wherein each expandable lobe extends radially from a respective offset point from a central longitudinal axis of the breast shield.

In some examples the lobes are defined by folds. Folded sections of the breast shield material create the lobes. The folds creating the lobes in FIG. 3 lie along or form lines which extend radially outwards from the centre of the breast shield/nipple tunnel. In the embodiment in FIG. 5, the folds creating or defining the lobes lie along or form lines which extend from a point which is offset from the centre of the breast shield/nipple tunnel. Each line extends outwards from the respective offset point. Each lobe is defined by a fold which extends from a different offset point, each offset point being at a location which is different to and offset from the centre of the breast shield/nipple tunnel.

In other words, for the plurality of expandable lobes, each expandable lobe extends radially from an offset point associated with that expandable lobe, wherein each offset point is in a different location. Each location is offset from the central longitudinal axis of the breast shield/nipple tunnel. Optionally, the plurality of offset points (associated with the plurality of expandable lobes) form a regular shape. For example, a triangle or a pentagon. Optionally, the regular shape may be centred around the central longitudinal axis of the breast shield.

Again, the different lobes may each be substantially similar in shape, as shown in FIG. 5. Alternatively, the different lobes may have different parameters, such as different amplitudes or thicknesses, and their lengths of longitudinal extension along the nipple tunnel may vary.

The breast shield may comprise a sealing extension 401 (not shown in FIG. 5). When the negative air pressure acts on the external walls of the nipple tunnel 109 of the breast shield according to FIG. 5, the lobes may expand radially and the aperture of the nipple tunnel may expand from a minimum aperture shown in FIGS. 5A and 5B to a maximum aperture. The maximum aperture may comprise a circle. Alternatively, the maximum aperture may comprise a convex circular triangle. The maximum aperture may be substantially more circular in shape than the minimum aperture. Therefore, the nipple tunnel approaches a circular aperture at maximum aperture. This may be the case for any embodiment of nipple tunnel described herein.

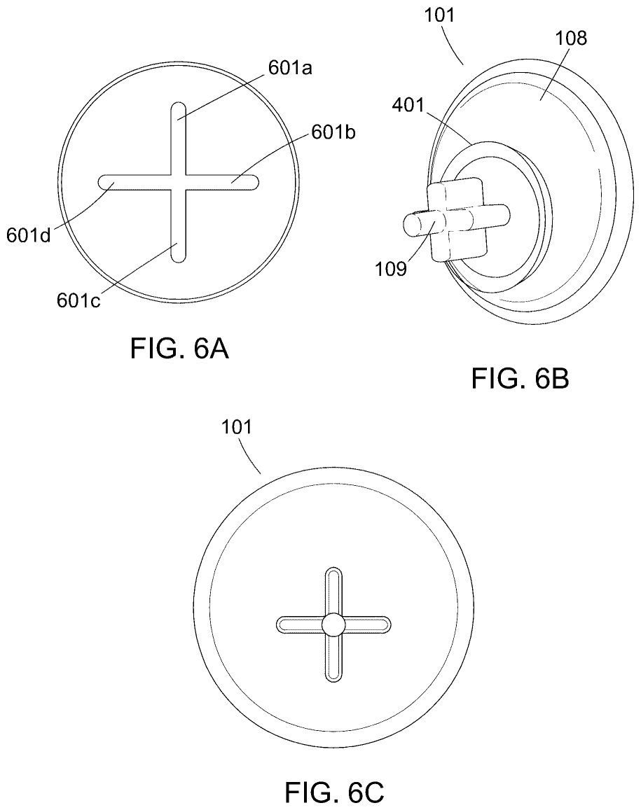

The embodiments of the breast shield described with reference to FIGS. 3 and 5 may be configured to comprise more than, or less than, three lobes. An embodiment configured to comprise four lobes is described as follows with reference to FIG. 6.

FIG. 6A shows a schematic of a geometry of a breast shield according to an embodiment of the present invention. In this embodiment, the nipple tunnel may comprise four lobes 601a, 601b, 601c, 601d. The four lobes may extend radially from a center of the nipple tunnel, and may be approximately perpendicular to one another, forming a ‘cross-shaped’ or ‘plus’ symbol-like arrangement when in the collapsed position. The lobes may each be substantially similar in shape, and/or have approximately the same size and depth, as shown in the embodiment in FIG. 6. Alternatively, the different lobes may have different parameters, such as different amplitudes or thicknesses.

In the expanded position, the aperture to the nipple tunnel approaches a maximum aperture. The maximum aperture may approach a circular aperture at maximum aperture. The maximum aperture may comprise a convex circular square, or a Reuleaux square.

In general, for a nipple tunnel comprising n lobes, the maximum aperture of the nipple tunnel may approximately form a Reuleaux polygon with n sides when the nipple tunnel is expanded to maximum aperture. In general, each of the n different lobes may be substantially similar in shape. Alternatively, some or all of the n different lobes may have different parameters, such as different amplitudes or thicknesses, and their lengths of longitudinal extension along the nipple tunnel may vary.

FIG. 6B shows a perspective view of the breast shield comprising the breast flange portion 108 and nipple tunnel 109 configured to comprise four separate lobes. FIG. 6C shows a different perspective view of the breast shield with the nipple tunnel according to the embodiment described above, viewed from the ‘inside’; the side which, in use, is configured to receive the user's breast. As can be seen, for example, in FIG. 6B, the lobes 601a, 601b, 601c, 601d may extend longitudinally along the length of the nipple tunnel.

The breast shield 101 according to FIG. 6 may comprise a sealing extension 401, as shown in FIG. 6B. The sealing extension is configured to provide a fluid seal for the region 402 (not shown in FIG. 6) surrounding the nipple tunnel 109, as described in detail with reference to FIG. 4. Any embodiment of the breast shield 101 described herein may comprise the sealing portion 401.

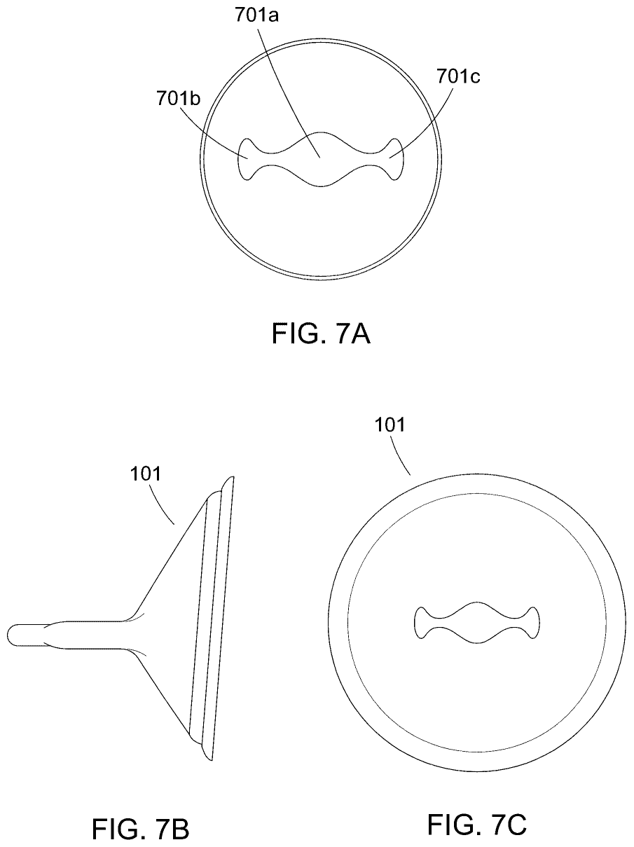

FIG. 6A shows a schematic of a geometry of the nipple tunnel 109 of a breast shield according to an embodiment of the present invention. In this embodiment, the nipple tunnel 109 may comprise a central circular or cylindrical portion 701a, and two approximately T-shaped lobes 701b, 701c extending radially from the central portion diametrically opposite one another. Each T-shaped lobe may comprise a narrow stem and a wider, approximately conical head to form a ‘cocktail glass’ shape, or T-shape. The nipple tunnel shown in FIGS. 7A-C comprising a central portion 701a and two ‘T-shaped’ lobes 701b, 701c may be arranged such that the opening to the nipple tunnel extends substantially in one dimension, for example laterally, or ‘horizontally’ with respect to the orientation of the breast shield. The nipple tunnel may be substantially ‘flat’, i.e. one dimensional, in a collapsed position. This can be seen in the perspective view of FIG. 7B. As will be appreciated by the skilled person, the T-shaped lobes 701b, 701c may be other suitable shapes, and the central portion 701a may be other suitable shapes.

FIG. 7C shows a perspective view of the breast shield with the nipple tunnel according to the embodiment described above, viewed from the ‘inside’; the side which, in use, is configured to receive the user's breast.

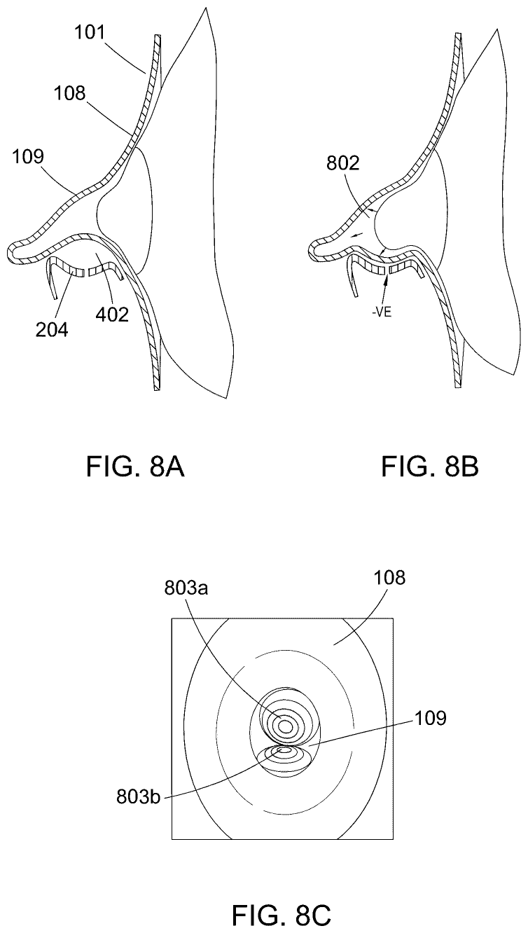

FIG. 8 shows an embodiment of the breast shield according to an embodiment of the present invention. The breast shield shown in FIGS. 8A-C comprises a breast flange portion 108 and nipple tunnel 109. The nipple tunnel 109 according to this embodiment comprises a single expandable lobe, extending along a longitudinal axis away from the breast shield. The cross-sectional area of the lobe may vary from a maximum at the aperture to a minimum at the end of the lobe furthest from the breast flange portion 108. In a collapsed position with air pumped from the nipple tunnel, shown in FIG. 8A, the variation in lobe cross-sectional area may be small such that the lobe diameter is at, or close to, the minimum along the whole lobe, with the volume of air present in the nipple tunnel minimised. As negative pressure is applied to the outside of the nipple tunnel, the lobe deforms or expands to the ‘expanded’ configuration shown in FIG. 8B. In the expanded position, the lobe may remain narrow at the end furthest from the breast flange portion 108, and may comprise a bulbous portion 802 closer to the breast shield, the bulbous portion having a larger cross-sectional area optionally similar to the aperture diameter. When the lobe expands in a radial direction from the collapsed position to the expanded position, the aperture of the nipple tunnel may dilate from a minimum to a maximum aperture, and vice versa.

FIG. 8C shows a perspective view of the breast shield according to the single-lobed embodiment of the nipple tunnel described above. The breast shield comprises a single piece, flexible diaphragm comprising a nipple tunnel 109. In the embodiment shown in FIGS. 8A-C, the single-lobed nipple tunnel may comprise a first expanding area 803a and a second expanding area 803b which expand radially as part of the nipple tunnel expansion when acted upon by negative pressure from outside the breast shield, thereby creating a bulbous portion 802. The first and second expanding areas 803a, 803b may be arranged so that when the nipple tunnel receives a user's nipple, the first expanding area 803a is positioned substantially on the ‘top’ of the nipple, and the second expanding area 803b is positioned substantially on the ‘bottom’ of the nipple. As used herein, the ‘bottom’ of the nipple corresponds to an area of the nipple closest to the milk container of the breast pump, and the ‘top’ of the nipple corresponds to an area diametrically opposite to the ‘bottom’, such that the nipple is arranged between the first and second expanding areas 803a, 803b.

In embodiments of the breast shield according to the present invention, the dilation of the nipple tunnel is radial rather than axial. The air pump actuates on the external walls of the nipple tunnel causing it to dilate radially around a central axis through the nipple tunnel. As a consequence, the nipple stays relatively in place during a pumping session, as opposed to being pulled in and out of the nipple tunnel as compared to traditional breast pumps. In other words, embodiments of the presently claimed breast shield encourage the nipple to dilate radially, rather than longitudinally, whilst also reducing internal air volume. A biomimetic motion is therefore achieved that allows a more natural breastfeeding experience and a more reassuring feeling for the user.

FIGS. 8A and 8B show an embodiment of the outer frame 204 configured for use with an embodiment of the nipple tunnel 109 according to FIG. 8. As shown, the outer frame may be arranged to define the outer boundary of the region 402 for application of negative pressure in particular around the expanding areas of the nipple tunnel, encouraging radial expansion of the expanding areas.

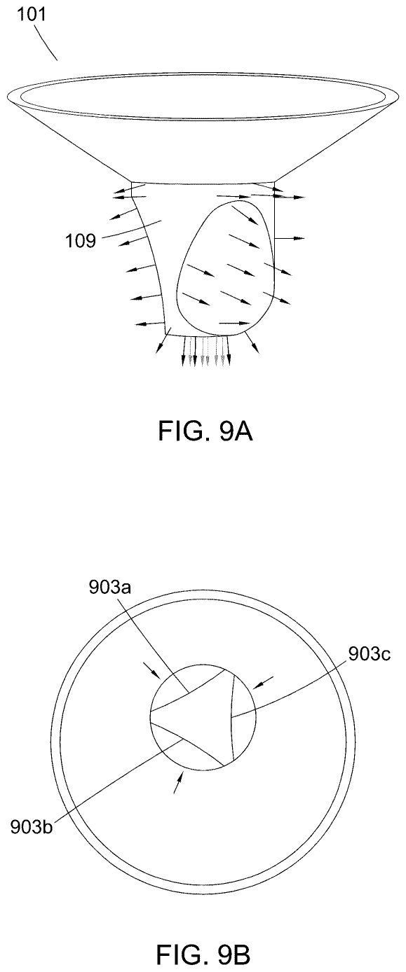

The embodiment of the breast shield according to the present invention described above with reference to FIGS. 8A-C may be configured such that the nipple tunnel comprises more than two expanding areas. An exemplary embodiment configured to comprise three expanding areas is described below with reference to FIG. 9.

FIGS. 9A and 9B show an embodiment of the breast shield according to the present invention comprising a nipple tunnel 109, wherein the nipple tunnel comprises a single lobe. In this embodiment, the single lobe is arranged in a rounded triangular prism shape, with three expanding areas 803a, 803b, 803c positioned around the circumferential surface of the nipple tunnel 109. Upon application of negative pressure, the expanding areas 903a, 903b, 903c expand in a radial direction, and the nipple tunnel may extend in a longitudinal direction. The nipple tunnel opening may dilate between a minimum aperture and a maximum aperture, and the nipple tunnel 109 may expand and contract between a collapsed position and an expanded position. This expansion is indicated by the arrows in FIG. 9A, whilst the arrows in FIG. 9B indicate the radial direction of deformation of the expanding areas 903a, 903b, 903c upon return to the collapsed position from the expanded position. The expanding areas 903a, 903b, 903c may comprise thinner material than the rest of the nipple tunnel 109 to encourage expansion in these areas.

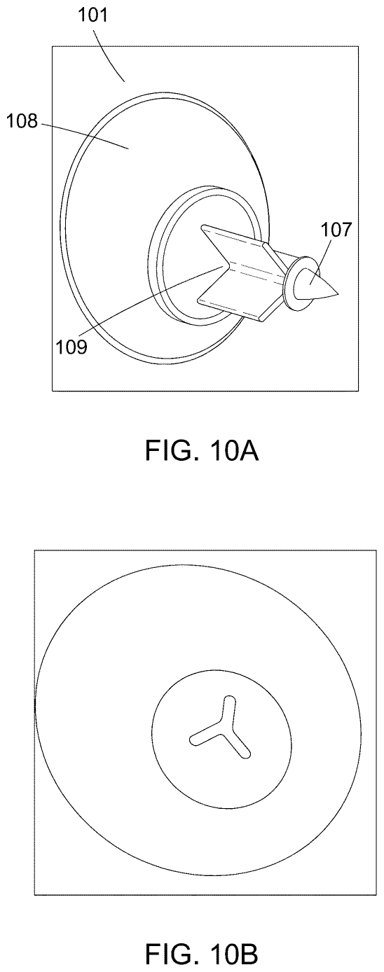

FIG. 10 shows an embodiment of the breast shield according to an embodiment of the present invention. The breast shield in FIGS. 10A and 10B is optically clear, and shows a nipple tunnel 109 comprising three expandable lobes similar to those of the embodiment described with reference to FIGS. 3A and 3B. As shown in FIGS. 10A and 10B, the lobes may extend longitudinally along the length of the nipple tunnel. Although the embodiment shown in FIG. 10 is optically clear, it will be understood that other embodiments may be optically opaque or translucent. Any embodiment according to the present invention may be optically clear, translucent or opaque.

The breast shield may comprise a non-return valve 107 at the end of the nipple tunnel furthest from the breast flange portion 108. In other words, the breast shield 101 may comprise a non-return valve 107 at one end of the nipple tunnel 109 and a breast flange 108 at another end of the nipple tunnel 109. The outer frame 204 (not shown in FIG. 10) may be configured such that the circular or cylindrical joint member 204d allows at least part of the non-return valve to pass through the centre of the joint member 204d, such that the joint member 204d permits passage of fluid out of the nipple tunnel 109 when the frame 204 and breast shield 101 are assembled for use with the breast pump 100 system.

The non-return valve 107 comprises a closing mechanism and is designed to allow fluid to pass in only one direction. Therefore, in some embodiment of the present invention breast milk is allowed to pass from the nipple tunnel 109 of the breast shield 101 to the milk container 104 where it is stored. The base level vacuum is drawn through the nipple tunnel and through the non-return valve. The negative pressure of the base level vacuum draws milk along the milk path from the nipple tunnel to the container. The valve is designed in shape so that when the pumping vacuum is applied the valve 107 is held closed. When fluid (i.e. milk and/or air) from the nipple tunnel 109 enters the valve, its pressure holds the closing mechanism open. However, milk or air flow from the container 104 to the nipple tunnel 109 is blocked due to the pressure of the milk or air on the non-return valve 107. The valve is designed so that if the fluid attempts to flow back through the non-return valve 107 in the wrong direction, a closing member of the closing mechanism is forced back over the entrance of the non-return valve 107 preventing any flow. This ensures that when the base level vacuum is not applied, e.g. after use, no fluid leaks from the milk container 104 back into the nipple tunnel 109 and towards the user.

The non-return valve reduces the volume of air on which the air pump is required to act. By preventing fluid, e.g. air and/or milk, from flowing from the milk container into the nipple tunnel, neither the base level vacuum nor the pumping vacuum is applied to the volume of fluid (i.e. the total volume comprising air and/or milk) in the milk container. A smaller volume requires less time and power to evacuate, hence the efficiency of the breast pump system is improved.

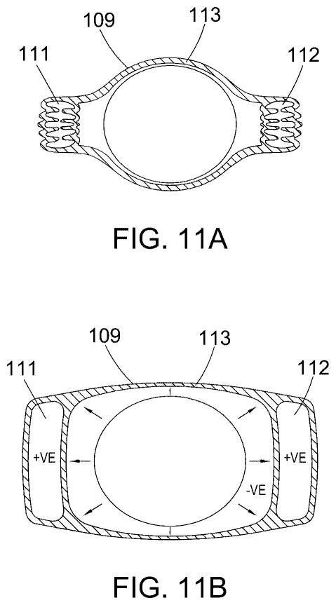

FIG. 11 shows an embodiment of the nipple tunnel 109 of the breast shield according to the present invention. Air pump 103 (not shown) may be used to generate positive pressure. FIG. 11 shows an exemplar embodiment of the breast shield 101 configured to use the positive pressure created by pump 103 to create negative pressure inside the nipple tunnel 109.

FIG. 11A shows an embodiment of the nipple tunnel 109 in the collapsed position. The nipple tunnel 109 comprises a single lobe 113, and a first inflation pocket 111 and a second inflation pocket 112 arranged laterally either side of the lobe 113, diametrically opposite one another. The inflation pockets 111, 112 are configured to expand in at least one of a radial, a lateral and a longitudinal direction upon application of positive pressure. In other words, the inflation pockets are configured to be inflated in at least one of a radial, a lateral and/or a longitudinal direction by positive pressure generated and applied by pump 103. Expansion of the inflation pockets 111, 112 either side of the lobe 113 causes deformation of the lobe, i.e. the nipple tunnel 109, such that the lobe, and hence the nipple tunnel 109, expands radially, as indicated by the red arrows in FIG. 11B. In general, application of positive pressure to the inflation pockets 111, 112 causes expansion of the lobe in at least one of a longitudinal, a radial and/or a lateral direction. FIG. 11B shows an embodiment of the nipple tunnel 109 in the expanded position. The increase in volume of the nipple tunnel 109 due to the radial expansion of the lobe in turn causes a negative pressure inside the nipple tunnel 109 (relative to atmospheric pressure), causing radial expansion of the user's nipple and encouraging milk expression.

The embodiment shown in FIG. 11 may be suitable for use in a breast pump 100 system with rotary diaphragm pumps, which may produce positive pressure more efficiently than negative pressure. The pumping system may be configured to apply a positive pressure to the inflation pockets 111, 112 periodically during a pumping cycle, causing the lobe (i.e. the nipple tunnel) to periodically expand and contract.

FIG. 11 shows an embodiment of the present invention where the breast shield for a breast pump comprises: a diaphragm comprising a nipple tunnel 109 for receiving a user's nipple, a breast flange portion, and an outer frame configured to define a region surrounding the nipple tunnel. The nipple tunnel comprises a single lobe 113 and at least one inflation pocket 111, 112 configured to receive positive pressure. The application of positive pressure to the at least one inflation pocket causes expansion of the lobe in at least one of a longitudinal, a radial and/or a lateral direction.

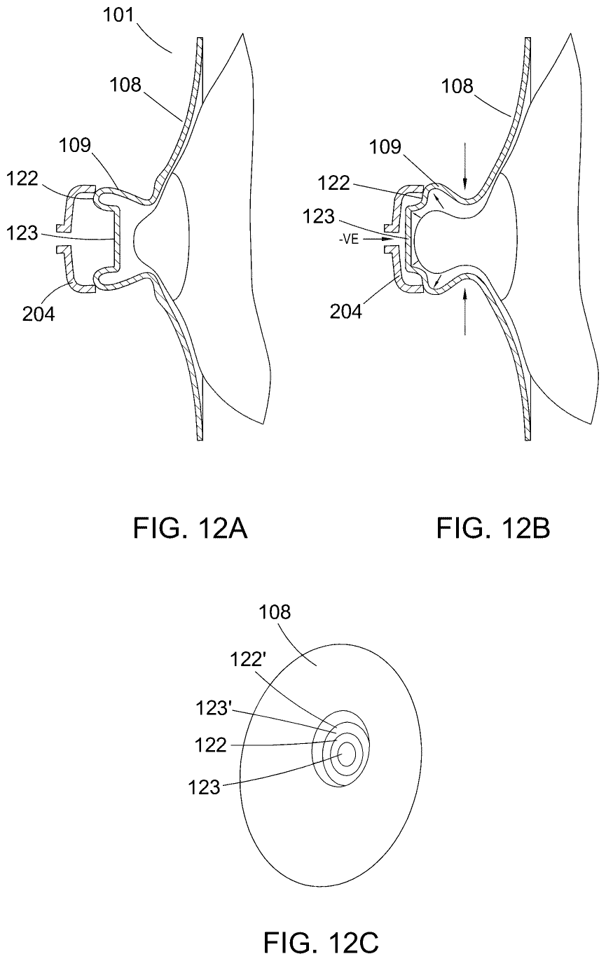

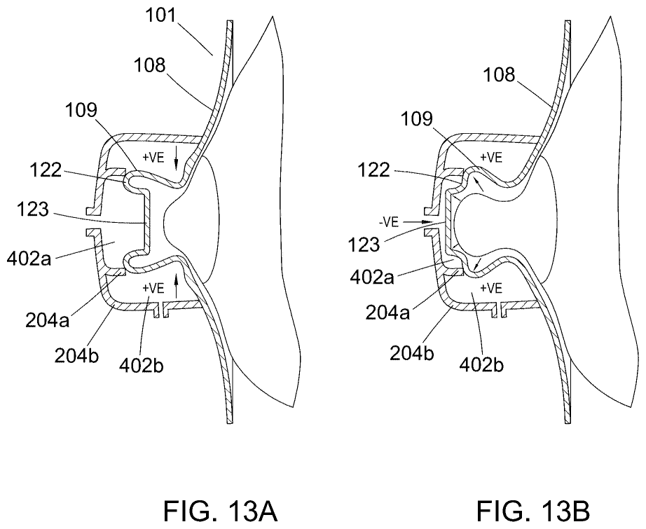

FIG. 12 shows an embodiment of the breast shield according to the present invention. The breast shield 101 in FIG. 12 comprises a passively collapsible nipple tunnel 109. The nipple tunnel 109 according to the embodiment of FIG. 12 may be considered to comprise a single lobe, and is substantially cylindrical in shape with a concave face at the end of the nipple tunnel 109 when in the ‘collapsed’ position, as shown in FIG. 12A. In the context of the embodiments shown in FIG. 12 and FIG. 13, the ‘end’ referred to is the end of the nipple tunnel 109 furthest from the breast flange 108. The concave face may comprise a trough 123 with a circular cross section, forming a ridge or ring 122 around the end circumference. The concave end of the nipple tunnel may comprise two or more concentric rings 122, 122′ and troughs 123, 123′, as shown in FIG. 12C. The nipple tunnel may be supported by an outer frame 204.

The thickness of the nipple tunnel wall at particular points may be configured such that, when acted on by the base level vacuum, the nipple tunnel 109 collapses around the nipple due to the pressure differential between the inside of the nipple tunnel 109 and the surrounding atmospheric pressure. In particular, the material of the nipple tunnel 109 may be thinned near the interface between the nipple tunnel 109 and the breast flange portion 108, such that the nipple tunnel is encouraged to collapse around the nipple at this point when the base level vacuum is applied, i.e., when a negative pressure is applied to the internal walls of the nipple tunnel 109. This is indicated by the green arrows in FIG. 12B. The at least one trough 123 and the at least one ridge 122 may be configured to have different thicknesses respectively to allow deformation of the single-lobed nipple tunnel 109 into the collapsed position shown in FIG. 12A, and expansion of the single-lobed nipple tunnel upon application of the pumping vacuum as indicated by the red arrows in FIG. 12B. The expansion of the nipple tunnel upon application of the pumping vacuum may be in at least one of a longitudinal and/or a radial direction.

As negative pressure is applied to the external walls of the nipple tunnel 109, the concave face is drawn towards outer frame 204, the at least one trough 123 and the at least one ridge 122 ‘unfold’ and the concave face becomes a convex face, as shown in FIG. 12B. Consequently, as external negative pressure is applied to the external walls of the nipple tunnel 109, the nipple tunnel 109 expands from the collapsed position to the expanded position, growing at least in length, i.e. at least longitudinally, creating a negative pressure inside the nipple tunnel 109 and drawing the nipple further into the nipple tunnel 109. The region between the outer frame 204 and the end of the nipple tunnel may be considered to be a first region, and the outer frame 204 may be considered to comprise a first extension. The pumping system may apply the external negative pressure to the external walls of the nipple tunnel in the first region periodically during a pumping cycle, causing the nipple tunnel to periodically expand and contract.

FIG. 13 shows an alternative configuration of the embodiment of the presently claimed breast shield described with reference to FIG. 12. The breast shield in FIG. 13 comprises an actively collapsible nipple tunnel 109. The nipple tunnel as shown in FIG. 12 can be considered to comprise a single lobe. As shown in FIG. 13, in this embodiment the outer frame comprises a first extension 204a to enclose a first region or chamber 402a, for application of negative pressure to the end of the nipple tunnel 109 comprising the concave face, and a second extension 204b to enclose a separate second region or chamber 402b, around the nipple tunnel 109. The second chamber 402b can be configured for application of positive or negative pressure to the nipple tunnel 109 in order to control the size of the nipple tunnel aperture and hence adjust the fit of the nipple tunnel 109 around the user's nipple.

The exemplar embodiment shown in FIG. 13 is configured for application of positive pressure in the second chamber 402b. Upon application of positive pressure, by pump 103 or alternatively by an additional pump, the nipple tunnel 109 is deformed such that it contracts radially around the user's nipple, conforming to the shape of the user's nipple and providing a tight fit. Therefore, when an external positive pressure is applied to the external walls of the nipple tunnel in the second region, the nipple tunnel contracts to a minimum aperture which conforms to the shape of the user's nipple. Negative pressure may be applied in the first chamber 402a to the external walls of the nipple tunnel optionally by pump 103, such that a negative pressure acts on the end of the nipple tunnel 123. This causes the concave face to become a convex face as described in detail with reference to FIG. 12, and the nipple tunnel 109 expands at least in a longitudinal direction. A resultant negative pressure is therefore generated inside the nipple tunnel 109, acting on the user's nipple, causing expansion of the user's nipple in at least a longitudinal direction and encouraging milk expression.

Embodiments of the breast shield according to FIG. 13 wherein the second chamber 402b is configured for the application of positive pressure to the nipple tunnel 109 may be suitable for use in a breast pump 100 system comprising rotary diaphragm pumps for efficient production of positive pressure. The pumping system may apply the external negative pressure to the external walls of the nipple tunnel in the first region periodically during a pumping cycle, causing the nipple tunnel to periodically expand and contract.

In alternative exemplar embodiments, the second chamber 402b may be configured for application of negative pressure to the nipple tunnel 109. In such embodiments, the application of negative pressure optionally by pump 103 may cause at least radial expansion of the single-lobed nipple tunnel 109. The first chamber 402a may be configured for application of negative pressure to the concave end of the nipple tunnel 109 as described above with reference to FIG. 13. A resultant negative pressure is therefore generated inside the nipple tunnel 109, acting on the user's nipple, causing expansion of the user's nipple in at least a longitudinal and a radial direction, and encouraging milk expression.

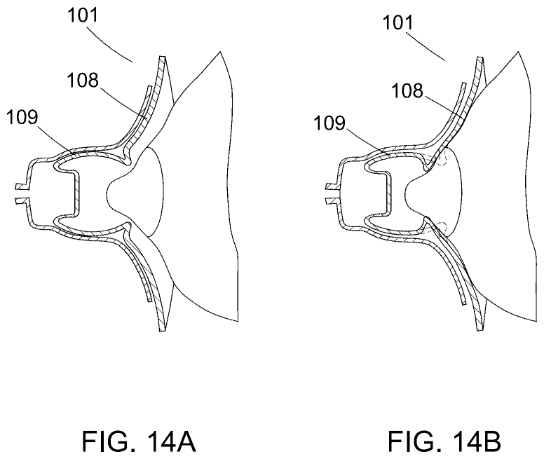

FIG. 14 shows an embodiment of the breast shield according to the present invention. The breast shield in FIG. 14 comprises a bi-stable nipple tunnel aperture. This embodiment of the breast shield 101 may provide an element of auto-sizing to the breast. The entrance to the nipple tunnel 109, i.e. the nipple tunnel aperture, is bi-stable in nature such that as the breast shield is pushed against, i.e. on to, the user's breast, the diameter of the nipple tunnel aperture may decrease in size from a first stable aperture until it clamps securely around the nipple at a second stable aperture. The second stable aperture may be smaller in diameter than the first stable aperture.

The nipple tunnel aperture sizes to the user as they press the breast pump device 100 onto the breast for use. The bi-stable nature of the geometry of the nipple tunnel 109 further provides a latching feeling.

FIG. 14A shows an embodiment of the breast shield before the breast pump 100 device is pressed onto the user's breast. In this embodiment the nipple tunnel is in a first stable position having a maximum aperture. FIG. 14B shows an embodiment of the breast shield once pressed onto the user's breast. In this embodiment the nipple tunnel is in a second stable position having a narrower aperture which forms a tight fit around the user's nipple. In FIG. 14B the original first stable position is indicated by dashed lines relative to the second stable position. The nipple tunnel 109 may be configured such that the volume inside the nipple tunnel is smaller in the second stable position than in the first stable position.

The nipple tunnel 109 may be considered to comprise a single lobe, and may additionally be configured to comprise a concave end as described with reference to FIGS. 12 and 13. The concave end may be configured to deform between a collapsed position and an expanded upon application of negative pressure, such that the nipple tunnel 109 expands at least in a longitudinal direction resulting in a negative pressure inside the nipple tunnel which act's on the user's nipple. As a result, the user's nipple expands at least in a longitudinal direction, thereby stimulating milk expression.

The bi-stable nipple tunnel aperture may be compatible with any embodiment of breast shield described herein.

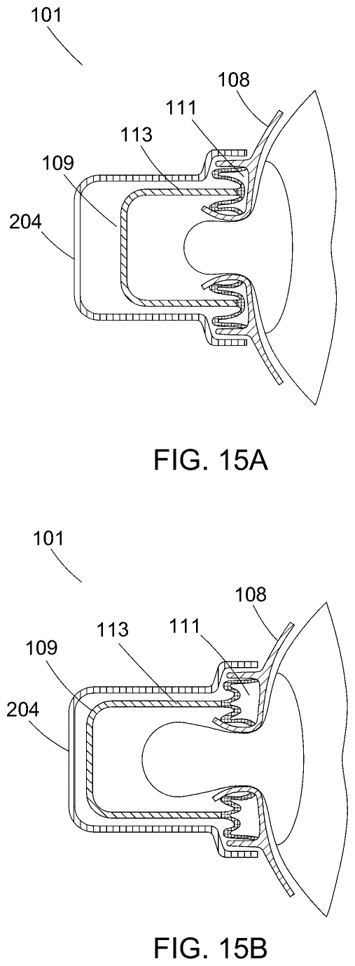

FIG. 15 shows an embodiment of the breast shield according to the present invention. The breast shield shown in FIG. 15 comprises a nipple tunnel 109 and a breast flange portion 108. FIG. 15 also shows the outer frame 204. The nipple tunnel 109 comprises a single lobe 113, and an inflation pocket 111 arranged between the lobe 113 and the breast flange 108. Generally, the nipple tunnel may comprise a single lobe and at least one inflation pocket. The at least one inflation pocket may be configured to receive positive pressure. The inflation pocket 111 may follow the circumferential shape of the lobe 113 at the base: for example, if the lobe is substantially cylindrical, the inflation pocket 111 may extend around the circumference of the lobe 113 at the base of the lobe in a circular tube or torus shape. As used herein, the term ‘base’ is intended to refer to the end of the lobe 113 closest to, optionally at the interface with, the breast flange 108. The inflation pocket 111 may also comprise a plurality of folds as shown in FIG. 15A which are folded when the inflation pocket is deflated, and unfold at least partially as the pocket is inflated. In alternative embodiments, the inflation pocket 111 may be a substantially smooth tube or bubble which stretches and expands upon inflation.

Similar to the embodiment of the nipple tunnel 109 described in detail with reference to FIG. 11, the inflation pocket 111 may be configured to inflate upon application of positive pressure. Therefore, application of positive pressure to the at least one inflation pocket may cause expansion of the lobe in at least one of a longitudinal, a radial, and/or a lateral direction. Inflation of the pocket increases the internal volume of the nipple tunnel 109, generating a negative pressure inside the nipple tunnel 109 which in turn causes expansion of the user's nipple and stimulates milk expression. The pumping system of the breast pump 100 system may be configured to apply a positive pressure to the at least one inflation pocket.

The inflation pocket 111 may be a single, continuous pocket around the whole circumference at the base of the lobe, or alternatively the nipple tunnel may comprise a plurality of inflation pockets positioned at various points around the circumference of the base of the lobe. In any case, the interface between the lobe and the inflation pocket, and the interface between the inflation pocket and the breast flange, may be configured to provide a fluid seal to enclose the fluid inside the nipple tunnel 109 such that negative pressure can be generated inside the nipple tunnel 109 upon inflation of the pockets.

The at least one inflation pocket 111 may be configured to expand in at least one of a longitudinal, a radial and a lateral direction upon application of positive pressure. In other words, the at least one inflation pocket 111 may be configured to be inflated in at least one of a longitudinal, a radial and/or a lateral direction by positive pressure generated and applied by a pump, for example pump 103. Expansion of the at least one inflation pocket 111 pushes the lobe 113 of the nipple tunnel 109 away from the user's breast, such that the nipple tunnel 109 expands in at least a longitudinal direction. FIG. 15A shows the nipple tunnel 109 in a collapsed position with the inflation pocket 111 not inflated; FIG. 15B shows the nipple tunnel 109 in an expanded position with the inflation pocket 111 at least partially inflated. The pumping system may apply the positive pressure to the at least one inflation pocket periodically during a pumping cycle, causing the lobe (i.e. the nipple tunnel) to periodically expand and contract.

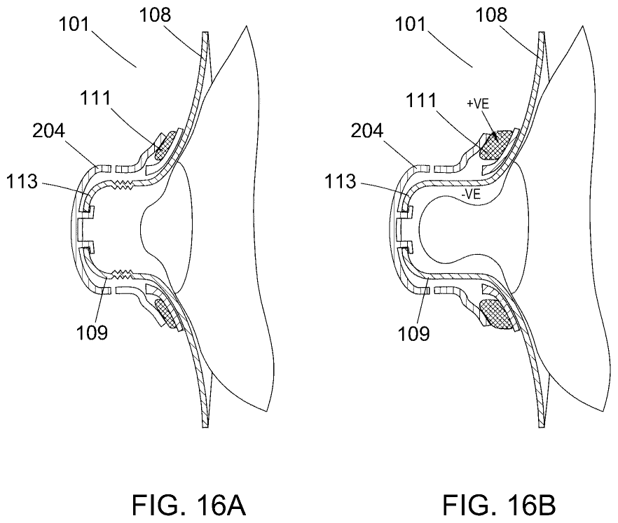

FIG. 16 shows an embodiment of the breast shield according to the present invention. FIG. 16 shows an alternative embodiment of the breast shield described with reference to FIG. 15. In FIG. 16, the at least one inflation pocket 111 may be arranged between the outer frame 204 and breast flange 108. The end of the lobe 113 of the nipple tunnel may be coupled to the frame 204. In the context of the embodiments shown in FIG. 16, the ‘end’ referred to is the end of the nipple tunnel 109 furthest from the breast flange 108.

The at least one inflation pocket 111 is configured as described in detail with reference to the inflation pocket of FIG. 15, i.e. the at least one inflation pocket is configured to expand in at least one of a radial, a lateral and/or a longitudinal direction when inflated by positive pressure generated and applied by a pump such as pump 103. Expansion of the at least one inflation pocket 111 pushes the frame 204 away from the user's breast. Since the end of the lobe 113 is coupled to the frame 204, when the frame is pushed away from the user's breast the nipple tunnel is also stretched in at least a longitudinal direction as the end moves away from the user's breast with the frame 204. This increases the volume inside the nipple tunnel, thereby generating a negative pressure inside the nipple tunnel (relative to atmospheric pressure) causing expansion of the user's nipple in at least a longitudinal direction and encouraging milk expression. During a pumping cycle, the pumping system may apply the positive pressure to the at least one inflation pocket periodically, causing the lobe (i.e. the nipple tunnel) to periodically expand and contract.

The embodiments shown in FIG. 15 and FIG. 16 may be suitable for use in a breast pump 100 system with rotary diaphragm pumps, which may produce positive pressure more efficiently than negative pressure.

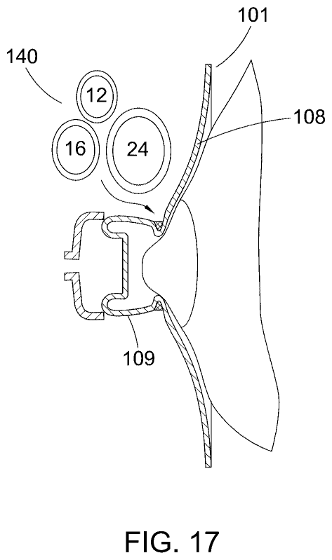

FIG. 17 shows an embodiment of the breast shield according to the present invention. The breast shield shown in FIG. 17 comprises sizing rings 140. Any one of the rings 140 may be applied to the nipple tunnel 109 at the base of the nipple tunnel 109, i.e. the end of the nipple tunnel 109 closest to the breast flange 108. The applied ring may define the size of the nipple tunnel aperture. Rings 140 may allow the breast shield to deform to any size required by the user, thereby improving the fit and performance of the breast pump 100 according to the user's needs. The rings enable a one-size breast shield 101 to be adjusted for different users' nipple sizes. Any embodiment of breast shield described herein may comprise sizing rings.

The breast shield 101 may comprise any one or more of a ring, a collar or other means of deforming the shape of the breast shield 101 configured to provide a custom fit to the user. The rings 140 may be used with a breast shield according to any embodiment described herein. The embodiment shown in FIG. 17 applies a ring 140 to a breast shield comprising a passively collapsible nipple tunnel such as that described with reference to FIG. 12, although the skilled person will understand that the rings may be applied to any other suitable configuration of breast shield.

The rings 140 shown in FIG. 17 comprise three exemplar sizes of ring: 12 mm diameter, 16 mm diameter and 24 mm diameter. Additional exemplar sizes may be provided to cater for a range of user nipple sizes. The rings may be formed of an elastic material, or a rigid material. The rings may be formed of silicone.

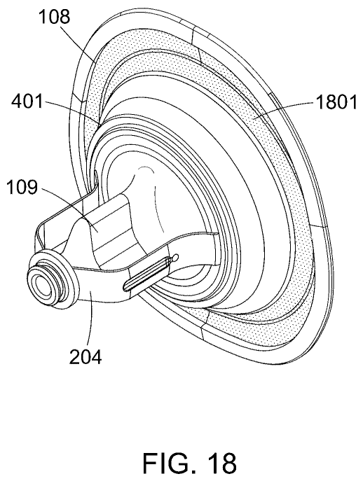

FIG. 18 shows an embodiment of the breast shield comprising a stiffening element 1801 according to the present invention. FIG. 18 shows a perspective view of the breast shield 101, outer frame 204, nipple tunnel 109 and stiffening element 1801. The stiffening element 1801 is shown in dotted lines. The outer frame 204 may be similar in structure as described in reference to FIGS. 4A to 4C. The stiffening element 1801 may be used in conjunction with any embodiment described herein.

The stiffening element 1801 may be ring shaped, extending around the entire circumference of the breast shield 101. The diameter of the stiffening element 1801 may be larger than the diameter of the outer frame 401 but smaller than the diameter of the outermost edge of the breast shield 101. That is, the stiffening element 1801 may be positioned on the breast shield 101 between the outer frame and the outermost edge of the breast shield. The stiffening element 1801 may be described as a stiffening collar.

In alternative embodiments, the stiffening element 1801 may extend around part of the circumference of the breast shield 101. The stiffening element 1801 may comprise a plurality of sub-stiffening elements. The plurality of sub-stiffening elements may be arc shaped extending around part of the circumference of the breast shield 101.

The stiffening element 1801 is configured to increase the stiffness of the breast shield around the circumference of the stiffening element. That is, increasing the stiffness of the breast shield as compared to the absence of the stiffening element 1801. The stiffening element may be made from any suitable material. For example, a plastic, elastomer or rubber such as silicone. The stiffening element may be made from a material harder and/or stiffer than the material of the rest of the breast shield 101. The stiffening element may be the same material as the rest of the breast shield 101 but of a greater thickness.

The stiffening element 1801 may be moulded as part of the breast shield 101 such that the breast shield is a single component. Alternatively, the stiffening element 1801 may be removable from the rest of the breast shield 101.

The stiffening element 1801 acts to stiffen the breast shield 101 around the stiffening element's circumference. This creates an air gap around the breast and breast shield. This air gap extends from the nipple tunnel (i.e. the nipple of the user) to the circumference of the stiffening element (i.e. the circumference of the breast of the user in line with the stiffening element). This air gap effectively widens out the effect of the pump's suction. Therefore, suction is applied to a larger area of the breast and is less localised on the nipple.

The sensation of the vacuum localised on the nipple can be uncomfortable for the user. The addition of the stiffening element 1801 allows for increased comfort. It also results in increased milk output for the user. In the event of a constant base level vacuum being applied, the stiffening ring results in a larger attachment force of the breast pump to the breast resulting in a more secure fit to the breast.

A removable stiffening element 1801 is advantageous as it allows the user to choose whether to localise the suction over the nipple or spread out the suction over more of the breast. For example, if the user is experiencing nipple sensitivity, they may choose to use the stiffening element 1801. Once their nipple sensitivity reduces, they may choose to remove the stiffening element 1801.

### Advantages

The wearable breast pump system may be configured to provide one or more of the following advantageous effects. The base level vacuum drawn through the nipple tunnel reduces the amount of air present in the overall pumping system, since there is a reduced level of air in the nipple tunnel compared to when a system without a base level vacuum is used. This means that the air pump acts on a smaller volume of air and is not required to work as hard in each pumping cycle, increasing the efficiency of the pumping system. In turn, this allows for improved battery lifetime.

In general, the breast shield according to an embodiment of the present invention is configured to conform to the shape of a nipple inserted into the nipple tunnel, thereby eliminating the need for the user to select the breast shield from a set of different sizes of breast shield to give the correct spacing. Therefore, breast shield according to an embodiment of the present invention is designed to fit all users. The conforming of the nipple tunnel to an inserted nipple may be automatic, i.e. without manual adjustment from the user, enabling quick and easy setup of the breast pump device for the user.

The breast shield provided results in a breast pump that can more easily be applied to breasts and nipples of differing shapes and sizes.

The base level vacuum system also reduces the likelihood of milk leaking from the breast pump during use. The base level vacuum ensures constant suction of the breast shield to the nipple and breast, meaning that contact is maintained even when the standard pumping vacuum is not being applied. This constant suction also allows the user to pump whilst bending or lying down since the breast shield is fixed to the user's breast.

The base level vacuum provided by an embodiment of the present invention provides a more precise base level vacuum and peak vacuum compared to previously known breast pumps.

The breast pump provided is a compact in-bra wearable device which is quiet in operation and may be controlled from a connected device. This provides a more discrete breast pump compared to previous known breast pumps. In particular, the user of the breast pump may wear the breast pump without the knowledge of people nearby, since both the shape, volume and ability to control the breast pump remotely from a connected device provide additional security to the user.

The connected device element allows for a user adjustable base level vacuum and/or pumping vacuum to improve comfort. The user can also adjust the base level vacuum and/or the pumping vacuum via an interface directly on the breast pump.

The breast pump provides improved comfort, since the base level vacuum improves comfort by decreasing rubbing and friction between the breast shield and nipple because the breast shield is tightly fitted to the breast.

The breast pump is easy to dry and clean since it is ensured that only certain aspects of the device are held in contact with the milk lactated from the breast.

The breast pump is easy and simple to assemble, disassemble and clean since the breast shield comprises a single flexible diaphragm. This is advantageous in particular over breast pumps comprising a separate breast shield and diaphragm.

The flexible diaphragm, made of a flexible material such as silicone, is removable and allows easy cleaning. A silicone part is durable, and can withstand high temperatures and chemicals used for cleaning. As such, a silicone breast shield provides a durable and sterilizable medical-grade part.

The breast shield according to the present disclosure may be configured for radial expansion of the nipple tunnel. Radial expansion of the nipple tunnel provides stimulation to the user's nipple via suction and forces in a radial direction. This is advantageous as it better mimics the action and stimulation provided to the user's nipple by a feeding child. This creates a more reassuring, realistic and comfortable experience for the user. It may also improve the efficiency of production of milk from a user's breast, since the motion better mimics the motion of a feeding infant.

Using a flexible breast shield with a nipple tunnel that is configured to expand radially improves dilation deformation by allowing a more circular aperture when fully opened. The ability for the nipple tunnel to conform to any user's nipple means that some embodiment of the present invention may provide a ‘one size fits all’ breast shield. Further, the breast shield is able to dynamically conform to the nipple and to adapt to any change of shape of the breast during a pumping session or during continuous multiple cycles of a pumping session.

The presently claimed breast shield and base level vacuum may facilitate a high peak pressure and cycle speed improvement during use of the breast pump.

The breast shield according to some embodiments of the present invention may provide improved ease of fitting the breast shield, and hence breast pump system to a user's breast.

Features of the above aspects can be combined in any suitable manner. It will be understood that the above description is of specific embodiments by way of aspect only and that many modifications and alterations will be within the skilled person's reach and are intended to be covered by the scope of the appendant claims.

The Disclosure Also Comprises the Following Clauses.
1. A breast shield for a breast pump, comprising:
   a flexible diaphragm comprising a nipple tunnel for receiving a user's nipple;
   wherein the breast shield is configured to form a seal with a user's breast and is configured such that:
   when an internal negative pressure is applied to internal walls of the nipple tunnel, the nipple tunnel contracts to a minimum aperture which conforms to the shape of the user's nipple.
2. The breast shield of clause 1, wherein the nipple tunnel comprises external walls, and wherein when an external negative pressure is applied to the external walls, the nipple tunnel expands radially up to a maximum aperture.
3. The breast shield of clause 1 or clause 2, wherein the nipple tunnel comprises a plurality of expandable lobes which extend radially outward from a central longitudinal axis of the breast shield.
4. The breast shield of clause 3, wherein the nipple tunnel comprises three lobes that dilate between the maximum aperture and the minimum aperture.
5. The breast shield of clause 4, wherein the three lobes each extend radially from a point offset from a central longitudinal axis of the breast shield.
6. The breast shield of clause 4 or clause 5, wherein the maximum aperture comprises a convex circular triangle.
7. The breast shield of clause 1 or clause 2, wherein the nipple tunnel comprises a single lobe, the single lobe comprising at least one expanding area.
8. The breast shield of clause 7, wherein the single lobe comprises a rounded triangular prism shape, with three expanding areas.
9. The breast shield of clause 3, wherein the nipple tunnel comprises four lobes that dilate between the maximum aperture and the minimum aperture.
10. The breast shield of clause 3, wherein the nipple tunnel comprises a central circular or cylindrical portion, and two T-shaped lobes.
11. The breast shield of any of clauses 2-10, wherein the nipple tunnel approaches a circular aperture at maximum aperture.
12. The breast shield of any preceding clause, wherein the flexible diaphragm is formed as a single piece, optionally by an injection moulded process or a compression moulded process, and optionally or additionally wherein the flexible diaphragm is formed of silicone.
13. The breast shield of any preceding clause, further comprising a non-return valve at one end of the nipple tunnel and optionally a breast flange portion at another end of the nipple tunnel.
14. The breast shield of any preceding clause, wherein the breast shield may be optically transparent, optically translucent or optically opaque.
15. The breast shield of any preceding clause, further comprising sizing rings.
16. A breast pump comprising:
   a pumping system; and
   the breast shield of any preceding clause,
   wherein the pumping system is configured to apply an internal negative pressure to the internal walls of the nipple tunnel.
17. The breast pump of clause 16, wherein the application of the internal negative pressure forms a vacuum seal between the breast shield and a user's breast.
18. The breast pump of clause 17, wherein the pumping system is further configured to apply an external negative pressure to external walls of the nipple tunnel.
19. The breast pump of clause 17 or clause 18, further comprising an outer frame configured to define a region surrounding the nipple tunnel.
20. The breast pump of clause 18 or clause 19, wherein the external negative pressure is greater in magnitude than the internal negative pressure.
21. The breast pump of any of clauses 18-20, wherein a level of suction between the breast shield and the user's breast is highest when both the internal negative pressure and the external negative pressure are applied to the nipple tunnel.
22. The breast pump of any of clauses 18-21, wherein the pumping system applies the external negative pressure to the external walls of the nipple tunnel periodically during a pumping cycle, causing expandable lobes of the nipple tunnel to periodically expand and contract between the minimum aperture and the maximum aperture.
23. The breast pump according to any of clauses 16-22, wherein the breast shield is removable from the breast pump.
24. The breast pump according to clause 23, wherein the breast shield may be removed and attached to the breast pump using a joint mechanism arranged on the outer frame, optionally wherein the joint mechanism is configured for a one press action optionally comprising audible and/or haptic feedback to indicate attachment and/or removal.
25. The breast pump according to any of clauses 16-24, wherein the breast pump is configured as a self-contained, in-bra wearable device.
26. A breast shield for a breast pump, comprising:
   a flexible diaphragm comprising a nipple tunnel for receiving a user's nipple; and
   an outer frame comprising a first extension configured to define a first region for application of negative pressure to external walls of the nipple tunnel;
   wherein the breast shield is configured to form a seal with a user's breast and is configured such that when an external negative pressure is applied to the external walls of the nipple tunnel in the first region, the nipple tunnel expands in at least a longitudinal direction.
27. The breast shield of clause 26, wherein when an internal negative pressure is applied to internal walls of the nipple tunnel the nipple tunnel contracts to a minimum aperture which conforms to the shape of the user's nipple.
28. The breast shield of clause 26 or clause 27, wherein the outer frame further comprises a second extension configured to define a second region for application of positive pressure to the external walls of the nipple tunnel.
29. The breast shield of clause 28, wherein when an external positive pressure is applied to the external walls of the nipple tunnel in the second region, the nipple tunnel contracts to a minimum aperture which conforms to the shape of the user's nipple.
30. The breast shield of any of clauses 25-29, wherein the nipple tunnel aperture is bi-stable such that as the breast shield is pushed onto the user's breast, a diameter of the nipple tunnel aperture decreases in size from a first stable aperture to a second stable aperture, wherein the second stable aperture is smaller in diameter than the first stable aperture.
31. a Breast Pump Comprising:
   a pumping system; and
   the breast shield of any of clauses 25-29,
   wherein the pumping system is configured to apply an external negative pressure to the external walls of the nipple tunnel in the first region.
32. The breast pump of clause 30, wherein the pumping system is further configured to apply an internal negative pressure to internal walls of the nipple tunnel.
33. The breast pump of clause 30, wherein the pumping system is further configured to apply an external positive pressure to the external walls of the nipple in the second region.
34. The breast pump of any of clauses 30-32, wherein the pumping system applies the external negative pressure to the external walls of the nipple tunnel in the first region periodically during a pumping cycle, causing the nipple tunnel to periodically expand and contract.
35. A breast shield for a breast pump, comprising:
   a diaphragm comprising a nipple tunnel for receiving a user's nipple;
   a breast flange portion; and
   an outer frame configured to define a region surrounding the nipple tunnel;
   wherein the nipple tunnel comprises a single lobe and at least one inflation pocket configured to receive positive pressure;

wherein application of positive pressure to the at least one inflation pocket causes expansion of the lobe in at least one of a longitudinal, a radial and/or a lateral direction.
36. The breast shield of clause 35, wherein the end of the lobe furthest from the breast flange portion is coupled to the outer frame.
37. The breast shield of clause 35 or clause 36, wherein the diaphragm is a flexible diaphragm.
38. The breast shield of any of clauses 35-37, wherein the at least one inflation pocket comprises a single inflation pocket extending around the circumference of the lobe at the end of the lobe closest to the breast flange portion, the single inflation pocket optionally comprising a torus shape.
39. The breast shield of any of clauses 35-37, wherein the at least one inflation pocket comprises a plurality of inflation pockets positioned at various points around the circumference of the lobe at the end of the lobe closest to the breast flange portion.
40. a Breast Pump Comprising:
   a pumping system; and
   the breast shield of any of clauses 35-39,
   wherein the pumping system is configured to apply a positive pressure to the at least one inflation pocket.
41. The breast pump of clause 40, wherein the pumping system applies the positive pressure to the at least one inflation pocket periodically during a pumping cycle, causing the lobe to periodically expand and contract.

1. A breast shield for a breast pump, comprising:
a flexible diaphragm comprising a nipple tunnel for receiving a user's nipple;
wherein the breast shield is configured to form a seal with a user's breast and is configured such that:
when an internal negative pressure is applied to internal walls of the nipple tunnel, the nipple tunnel contracts to a minimum aperture which conforms to the shape of the user's nipple, wherein the nipple tunnel comprises a plurality of expandable lobes wherein each expandable lobe extends radially from a respective offset point from a central longitudinal axis of the breast shield.

2. The breast shield of claim 1, wherein the nipple tunnel comprises external walls, and wherein when an external negative pressure is applied to the external walls, the nipple tunnel expands radially up to a maximum aperture.

3. The breast shield of claim 2, wherein the nipple tunnel comprises three lobes that dilate between the maximum aperture and the minimum aperture.

4. (canceled)

5. The breast shield of claim 1-or wherein the nipple tunnel comprises a single lobe, the single lobe comprising at least one expanding area.

6. The breast shield of claim 5, wherein the single lobe comprises a rounded triangular prism shape, with three expanding areas.

7. The breast shield of claim 2, wherein the nipple tunnel comprises four lobes that dilate between the maximum aperture and the minimum aperture.

8. The breast shield of claim 2, wherein the nipple tunnel comprises a central circular or cylindrical portion, and two T-shaped lobes.

9. The breast shield of claim 2, wherein the nipple tunnel approaches a circular aperture at maximum aperture.

10. The breast shield of claim 1, further comprising a non-return valve at one end of the nipple tunnel and optionally a breast flange portion at another end of the nipple tunnel.

11. (canceled)

12. The breast shield of claim 1, further comprising sizing rings.

13. The breast shield of claim 1, wherein the nipple tunnel aperture is bi-stable such that as the breast shield is pushed onto the user's breast, a diameter of the nipple tunnel aperture decreases in size from a first stable aperture to a second stable aperture, wherein the second stable aperture is smaller in diameter than the first stable aperture.

14. The breast shield of claim 1, further comprising at least one inflation pocket configured to receive positive pressure, wherein application of positive pressure to the at least one inflation pocket causes expansion of the lobe in at least one of a longitudinal, a radial and/or a lateral direction.

15. The breast shield of claim 14, wherein the at least one inflation pocket comprises either:
a single inflation pocket, the single inflation pocket optionally comprising a torus shape; or
a plurality of inflation pockets positioned at various points around the circumference of the lobe.

16. (canceled)

17. (canceled)

18. A breast pump comprising:
a pumping system; and
the breast shield of any preceding claim,
wherein the pumping system is configured to apply an internal negative pressure to the internal walls of the nipple tunnel.

19. The breast pump of claim 18, wherein the application of the internal negative pressure forms a vacuum seal between the breast shield and a user's breast, wherein the pumping system is further configured to apply an external negative pressure to external walls of the nipple tunnel, and wherein the external negative pressure is greater in magnitude than the internal negative pressure.

20. (canceled)

21. The breast pump of claim 18, further comprising an outer frame configured to define a region surrounding the nipple tunnel.

22. The breast pump of claim 19, wherein the pumping system applies the external negative pressure to the external walls of the nipple tunnel periodically during a pumping cycle, causing expandable lobes of the nipple tunnel to periodically expand and contract between the minimum aperture and the maximum aperture.

23. The breast pump according to claim 21, wherein the breast shield is removable from the breast pump, wherein the breast shield may be removed and attached to the breast pump using a joint mechanism arranged on the outer frame, optionally wherein the joint mechanism is configured for a one press action optionally comprising audible and/or haptic feedback to indicate attachment and/or removal.

24. (canceled)

25. The breast pump of claim 18, wherein the outer frame comprises:
a first extension configured to define a first region for application of negative pressure to external walls of the nipple tunnel; and
a second extension configured to define a second region for application of positive pressure to the external walls of the nipple tunnel.

26. The breast pump of claim 18, wherein the pumping system applies the positive pressure to at least one inflation pocket periodically during a pumping cycle, causing the lobe to periodically expand and contract.
